(19) United States
(12) Patent Application Publication
(10) Pub. No.: US 2024/0150602 A1
(43) Pub. Date: May 9, 2024

(54) ANTIMICROBIAL AND/OR ANTIVIRAL COATINGS

(71) Applicant: CODIKOAT LTD, Haverhill, Cambridgeshire (GB)

(72) Inventors: Mohammad Javad MOHSENI, Haverhill, Cambridgeshire (GB); Reza Saberi MOGHADDAM, Haverhill, Cambridgeshire (GB); Payam NAHAVANDI, Haverhill, Cambridgeshire (GB); Atif AZIZ; Mohamed Basel BAZBOUZ, Haverhill, Cambridgeshire (GB)

(73) Assignee: CODIKOAT LTD, Haverhill, Cambridgeshire (GB)

(21) Appl. No.: 18/281,645

(22) PCT Filed: Mar. 11, 2022
(86) PCT No.: PCT/EP2022/056417

§ 371 Date: Sep. 12, 2023

(30) Foreign Application Priority Data
Mar. 12, 2021 (GB) 2103492.1
Dec. 23, 2021 (GB) 2118910.5

(51) Int. Cl. C09D 11/52; C09D 7/61; H01G 9/10; H01L 31/0216; H01M 50/119; H01M 50/124; H10K 30/88

(52) U.S. Cl. CPC C09D 11/52; C09D 7/61; H01G 9/10; H01L 31/02167; H01M 50/119; H01M 50/1245; H10K 30/88

(57) ABSTRACT

The present invention relates to antimicrobial and antiviral coatings. Described is an insulator substrate having an antimicrobial/antiviral layer on a surface thereof in which the antimicrobial/antiviral layer comprises a conductive material connected to a power supply. The power supply and conductive material together induce a voltage across the antimicrobial/antiviral layer such that a capacitor structure is formed on the substrate surface.

### FIELD OF THE INVENTION

The present invention relates to a substrate having an antimicrobial and/or antiviral coating. The invention also relates to an insulator substrate having an antimicrobial and/or antiviral layer thereon, said layer comprising a charged conductive material. The invention further relates to a method of manufacturing an antimicrobial and/or antiviral layer comprising a charged conductive material on a surface of an insulator substrate. The invention further relates to a method of manufacturing a coating of the invention. The invention also relates to the use of printing, lithography, ALD, CVD and similar techniques to deposit antimicrobial and/or antiviral material on a substrate. The invention further relates to uses of the coatings and substrates of the invention.

### BACKGROUND OF THE INVENTION

Germs such as viruses, bacteria and fungi can be readily transmitted from one host to another. Germs do not move themselves but rely on people, the environment, and/or medical equipment to move. Transmission can happen as a result of contact with a contaminated surface, sprays and splashes and inhalation of droplets carrying germs.

A large number of germs are harmless and even helpful to humans and animals. However, others are harmful and can cause infections. In such cases, the prevention of the spread of germs plays an important role in protecting vulnerable subjects.

Frequently touched points are a considerable vector for transmission of infectious diseases, and currently, there is no effective solution for this. The antimicrobial and antiviral properties of a number of metals such as silver, copper, zinc, gold, titanium, aluminium, tin, iron, arsenic, lanthanum, and molybdenum are known. Further, nanoparticles in the form of metal compounds such as titanium oxide, zinc oxide, aluminium oxide, copper oxide, silicon oxide and silver oxide, as well as other silver, copper and zinc compounds have been reported to elicit bactericidal properties through the generation of reactive oxygen species (ROS) that are able to target physical structures, metabolic pathways, and DNA synthesis of prokaryotic cells leading to cell death (Gold, K. et al. Antimicrobial Activity of Metal and Metal-Oxide Based Nanoparticles. Adv. Therap., 1(3) 1700033 (2018)). However, these nanoparticles can take hours to destroy microorganisms and their efficiency against viruses has not been established. On the other hand, disinfecting agents only last a few minutes and do not provide a reliable protection.

Therefore, there remains a need to provide new antiviral and antimicrobial agents that can efficiently kill microbes and viruses in a timely manner.

The present invention produces a fast-acting and long-lasting solution which can inactivate viruses, bacteria and fungi on a wide range of surfaces and last the lifetime of the product.

### SUMMARY OF THE INVENTION

The present invention relates to a substrate having an antimicrobial and/or antiviral coating. The coating may be a layer, said layer comprising a charged conductive material. The conductive material is connected to a power supply, and the power supply and the conductive material are arranged such that the power supply is capable of inducing a voltage across the layer, such that a capacitor structure is formed on a surface of the substrate.

Here, presented for the first time, is an insulator substrate having an antimicrobial and/or antiviral layer thereon, said layer comprising a charged conductive material that can efficiently kill microbes and viruses in a timely manner Using electricity and water electrolysis to provide rapidly deployable, fast-acting and long-lasting protection, this technology is capable of inactivating viruses such as coronavirus in seconds.

The invention relates to an insulator substrate having an antimicrobial and/or antiviral layer thereon, said layer comprising a charged conductive material. The conductive material is connected to a power supply, and the power supply and the conductive material are arranged such that the power supply is capable of inducing a voltage across the film layer, such that a capacitor structure is formed on a surface of the substrate. The conductive material is charged because the material, when connected to a power supply, behaves as capacitor electrodes.

In an embodiment, the conductive material is arranged into at least two, or a plurality of, strips on a surface of the substrate, wherein the strips are spaced apart from each other, and wherein the strips are alternately connected to positive and negative connections of the power supply, so as to induce a voltage across the film layer between adjacent strips.

In an embodiment, the conductive material is arranged into at least two, or a plurality of, strips on a surface of the substrate, wherein the strips are spaced apart from each other, and wherein the strips are alternately a photocathode and a photoanode, so as to induce a voltage across the film layer between adjacent strips.

In an embodiment, the voltage induced by the power supply is in the range of 10 mV to 50 V. In an embodiment, the voltage induced by the power supply is in the range of 1V to 10V, preferably in a range of between 1V and 5V.

In an embodiment, the conductive material comprises at least one metal, optionally selected from silver, aluminium, zinc, copper, titanium, chromium, iron, platinum, palladium, tungsten, and gold. It will be appreciated that where the conductive material comprises more than one metal, the conductive material comprises multilayer combinations and/or alloys. In an embodiment, the conductive material comprises a metal oxide, optionally selected from nickel-, cobalt- and iron-based oxides, indium tin oxide and fluorinated tin oxide and mixtures and combinations thereof. In an embodiment, the conductive material comprises a doped metal oxide, optionally wherein the doped metal oxide is aluminium doped zinc oxide. In an embodiment, the conductive material comprises a non-metal such as graphite, graphene or carbon, for example carbon nanostructures such as carbon nanotubes. In a further embodiment, the conductive material comprises a mixture, or combination, of metal and non-metal components, such as silver in combination with graphene, for example. A combination of metal and non-metal components may be as combined compositions or as one or more discrete layers.

In an embodiment, the conductive material consists of at least one metal, optionally selected from silver, aluminium, zinc, copper, titanium, iron, chromium, platinum, palladium, tungsten, and gold and mixtures, combinations and alloys thereof. In another embodiment, the conductive material consists of a metal oxide, optionally selected from nickel-, cobalt- and iron-based oxides, indium tin oxide and fluorinated tin oxide and mixtures and combinations thereof. Examples include NiCo₂O₄, doped Co₃O4, SrFeO₃, (Ni, Fe)Ox.

In another embodiment, the conductive material consists of a doped metal oxide, optionally wherein the doped metal oxide is aluminium doped zinc oxide. In another embodiment, the conductive material consists of a non-metal such as graphite, graphene or carbon, for example carbon nanostructures such as carbon nanotubes.

In an embodiment, the conductive material comprises nanoparticles.

Where photocathode and a photoanode strips are present, one or more components of the conductive material may comprise photoactive metal nanoparticles, metal oxide nanoparticles, metal sensitised with photoactive dyes, and/or metal oxides sensitised with photoactive dyes, together with mixtures, combinations and alloys thereof.

In an embodiment, the strips are made of a single layer or multilayers of conductive material.

In an embodiment, the antimicrobial and/or antiviral layer is substantially transparent or semi-transparent, or has an overall transparency of between about 50% and 99% in the visible spectrum. Optionally, the antimicrobial and/or antiviral layer is made of nickel-, cobalt- and iron-based oxides, indium tin oxide, fluorinated tin oxide, aluminium doped zinc oxide. Alternatively, in an embodiment, the antimicrobial and/or antiviral layer is substantially opaque. Optionally, the antimicrobial and/or antiviral layer is made of silver, aluminium, zinc, copper, titanium, iron, or gold.

In an embodiment, the distance between each adjacent strips of conducting material on the surface of the substrate independently is in the range of 0.5 μm to 1 cm, preferably in the range of 2 μm to 300 μm, more preferably in the range of 2 μm to 200 μm.

In an embodiment, the width of each strip of conducting material may be about 2 to 100 times smaller than the distance between the conductive material strips. For example, the width of each strip independently may be in the range of 0.5 μm to 1 cm, preferably in the range of 2 μm to 500 μm, more preferably in the range of 2 μm to 300 μm. In a particular embodiment, the width of each strip of conducting material is between about 10 nm and 50 μm, preferably less than about 5 μm. In a particular example, the width of the conducting strips may be in a range of between about 1 μm and about 5 μm. In another example, the width of the conducting strips may be in a range of between about 10 μm and about 200 μm.

In an embodiment, the thickness (or depth) of each strip of conducting material independently may be in the range of 5 nm to 1000 μm, preferably in the range of 500 nm to 500 μm, more preferably in the range of 1 μm to 100 μm. In a particular example, the thickness of the conducting strips may be in a range of between about 20 nm and about 500 nm. In another example, the width of the conducting strips may be in a range of between about 1 μm and about 10 μm.

While the conductive material may be laid out in strips of any suitable patterns, a particularly suitable pattern is an interdigitated array in which the strips connected to one output of the power source are arranged in a finger-like pattern and the fingers fit in between opposing “fingers” provided by the strips connected to the other output of the power source.

In an embodiment, the antimicrobial and/or antiviral layer further comprises a hygroscopic component. Preferably, the hygroscopic component is selected from rock salt, silica sand, silica gel, CaCl₂, a hygroscopic polymer, or a glycosaminoglycan. In an embodiment, the hygroscopic component comprises an aqueous component, preferably water.

In an embodiment, the antimicrobial and/or antiviral layer is partially or fully covered by a protective layer. Optionally, the protective layer is selected from epoxy or any non-conductive metal oxide such as aluminium oxide or silicon dioxide.

In an embodiment, the surface of the insulator substrate beneath the antimicrobial and/or antiviral layer has been pre-treated with oxygen plasma or UV ozone, such that the surface tension of the surface of the insulator substrate is reduced.

In an embodiment, the power supply is a solar cell. Preferably, the solar cell is an organic or amorphous silicon solar cell, preferably wherein the solar cell is an OPV (organic photovoltaic), a DSSC (Dye-sensitised solar cells), a copper indium gallium selenide (CIGS) solar cell, or a flexible silicon solar cell.

In another embodiment, the power supply is a battery. Preferably, the battery has a thickness in the range of 1 mm to 10 mm, or in the range of 10 μm to 100 μm.

In an embodiment, the conductive material is connected to the power supply by a wire. In an embodiment, the wire comprises: a metal, a conductive metal oxide, a doped metal oxide, and/or graphene or carbon, for example carbon nanostructures such as carbon nanotubes.

In an embodiment, the insulator substrate is flexible, optionally wherein the insulator substrate is a polymeric adhesive film, fabric, plastic, paper, fibres, or amorphous silicon. In another embodiment, the insulator substrate is non-flexible, optionally wherein the insulator substrate is glass, plastic, or ceramic. For the avoidance of doubt, the insulator, by virtue of its definition, is non-conductive.

The present invention also relates to an article of manufacture comprising the insulator substrate defined herein. In an embodiment, the article of manufacture is selected from an electronic device such as a tablet or mobile phone, a touchscreen, a keypad, a keyboard, a button, a handle, a screen protector, a piece of furniture such as a seat, a table, an arm rest, a toilet seat, a cup, a worktop, and an appliance, such as a kitchen appliance, a door plate, a water tap, a car steering wheel, a car key, food packaging, stationary, and a bank note.

The invention further relates to the use of printing, atomic layer deposition or lithography such as photolithography, preferably inkjet or screen printing, in the manufacture of an antimicrobial and/or antiviral layer comprising a charged conductive material as defined herein on a surface of an insulator substrate, or of an antimicrobial and/or antiviral layer comprising a charged conductive material as defined herein on a surface of an article of manufacture as defined herein.

The invention also relates to the use of laser ablation or cutting system in the manufacture of an antimicrobial and/or antiviral layer comprising a charged conductive material as defined herein on a surface of an insulator substrate, or of an antimicrobial and/or antiviral layer comprising a charged conductive material as defined herein on a surface of an article of manufacture as defined herein, wherein laser ablation or cutting system is used to remove strips from a commercially existing conductive film to create strips of conducting material.

The present invention further relates to a method of making an insulator substrate as defined herein or an article of manufacture as defined herein.

The present invention also relates to a method of making a substrate having an antimicrobial and/or antiviral film layer thereon, said film layer comprising a conductive material, wherein the method comprises the steps of:
a) providing a precursor substrate and a conductive material ink, wherein the precursor substrate is made of an insulator material,
b) printing the conductive material ink on a surface of the precursor substrate.

In an embodiment, the conductive material ink comprises a metal, a metal oxide, and/or a doped metal oxide and/or mixtures and alloys thereof, and a solvent. Optionally, the metal and/or metal oxide is selected from silver, nickel-, cobalt- and iron-based oxides, indium tin oxide, fluorinated tin oxide, aluminium doped zinc oxide, aluminium, zinc, copper, titanium, iron, nickel, cobalt, tungsten, palladium, platinum, and gold. In an embodiment, the conductive material ink comprises a non-metal such as graphite, graphene or carbon and mixtures and combinations thereof, for example carbon nanostructures such as carbon nanotubes, and a solvent. In an embodiment, the conductive material ink comprises nanoparticles. In an embodiment, the conductive material ink comprises photoactive metal nanoparticles, metal oxide nanoparticles, metal sensitised with photoactive dyes, and/or metal oxides sensitised with photoactive dyes, and a solvent. In an embodiment, the solvent is ethanol.

In an embodiment, step b) of the method further comprises printing the conductive material ink to form at least two, or a plurality of, strips of conductive material on a surface of the precursor substrate, wherein the strips are spaced apart from each other as defined and described herein. In an embodiment, step b) of the method is repeated to form multiple layers of conductive material.

In an embodiment, a photolithography process may be used to produce a photomask of the required pattern before printing the conductive material ink on the surface of the precursor substrate.

In an embodiment, step a) of the method further comprises treating the precursor substrate with oxygen plasma or UV ozone, such that the surface tension of the surface of the insulator substrate is reduced.

The present invention also relates to a method of making a substrate having an antimicrobial and/or antiviral layer thereon, said layer comprising a conductive material, wherein the method comprises the steps of:
a) providing a precursor substrate made of an insulator material and a substrate coated with a layer of conductive material,
b) removing strips of conductive material using laser ablation or cutting system, thereby forming strips of conducting material on the surface of the substrate, wherein the strips are spaced apart from each other.
c) adhering the laser-cut strips of conductive material on the precursor substrate made of an insulator material.

In an embodiment, the precursor substrate is an aluminium tape.

In an embodiment, the distance between each adjacent strips on the surface of the precursor substrate independently may be in the range of 0.5 μm to 1 cm, preferably in the range of 2 μm to 200 μm, more preferably in the range of 2 μm to 300 μm.

In an embodiment, the width of each strip is between about 2 to 100 times smaller than the distance between strips. For example, each strip may independently have a width in the range of 0.5 μm to 1 cm, preferably in the range of 2 μm to 500 μm, more preferably in the range of 2 μm to 300 μm. In a particular example, each strip may independently have a width in the range of between about 1 μm to about 5 μm. In another example, each strip may independently have a width in the range of between about 10 μm to about 200 μm.

In an embodiment, the thickness of each strip independently is in the range of 5 nm to 1000 μm, preferably in the range of 500 nm to 500 μm, more preferably in the range of 1 μm to 100 μm. A suitable thickness is also about 10 nm to 200 nm. In a particular example, each strip may independently have a thickness (or depth) in the range of between about 20 nm to about 500 nm. In another example, each strip may independently have a thickness (or depth) in the range of between about 1 μm to about 10 μm.

In an embodiment, the method described herein further comprises printing or placing a power supply on a surface of the precursor substrate, wherein the power supply and the conductive material are arranged such that the power supply is capable of inducing a voltage across the antimicrobial and/or antiviral layer on a surface of the precursor substrate. In an embodiment, the power supply is a solar cell or a battery. In an embodiment, the power supply is a solar cell, optionally wherein the solar cell is an organic or amorphous silicon solar cell, preferably wherein the solar cell is an OPV (organic photovoltaic), a DSSC (Dye-sensitised solar cells), a copper indium gallium selenide (CIGS) solar cell, or a flexible silicon solar cell. In another embodiment, the power supply is a battery, optionally wherein the battery has a thickness in the range of 10 μm to 100 μm.

In an embodiment, the method described herein further comprises printing a wire, wherein the wire connects each strip of conductive material to the power supply. In an embodiment, the wire comprises: a metal, a conductive metal oxide, a doped metal oxide, and/or graphite, graphene or carbon, for example carbon nanostructures such as carbon nanotubes as described and defined herein.

In an embodiment, the method described herein further comprises layering a hygroscopic component on the precursor substrate. In an embodiment, the hygroscopic component is selected from rock salt, silica sand, silica gel, CaCl₂, a hygroscopic polymer, or a glycosaminoglycan. In an embodiment, the hygroscopic component comprises an aqueous component, preferably water.

In an embodiment, the method described herein further comprises depositing a partial or full protective layer on the conductive material. In an embodiment, the protective layer is deposited by physical vapour deposition, chemical deposition such as ALD, or printing. In an embodiment, the protective layer is selected from epoxy or any non-conductive metal oxide such as aluminium oxide or silicon dioxide, as defined and described herein.

In an embodiment, the antimicrobial and/or antiviral layer is substantially transparent, semi-transparent, or has an overall transparency of between about 50% and 99% in the visible spectrum. In an example, the antimicrobial and/or antiviral layer may be made of made of indium tin oxide, fluorinated tin oxide, aluminium doped zinc oxide. In another embodiment, the antimicrobial and/or antiviral layer is substantially opaque, optionally wherein the antimicrobial and/or antiviral layer is made of silver, aluminium, zinc, copper, titanium, iron, or gold.

In an embodiment, the substrate precursor is flexible, optionally wherein the substrate precursor is a polymeric adhesive film, fabric, plastic, paper, fibres, or amorphous silicon. In another embodiment, the substrate precursor is non-flexible, optionally wherein the substrate precursor is glass, plastic or ceramic.

The invention also relates to a method of depositing an insulator substrate as defined herein or an article of manufacture as defined herein.

The invention further relates to the use of the insulator substrate as defined herein, or the article of manufacture as defined herein, as an antimicrobial agent. The term “antimicrobial” encompasses bacteria and fungi.

The invention also relates to the use of the insulator substrate as defined herein, or the article of manufacture as defined herein, as an antiviral agent.

The invention also relates to the use of the insulator substrate as defined herein, or the article of manufacture as defined herein, for inactivating a germ.

The invention also relates to the use of an antimicrobial and/or antiviral layer for inactivating a germ,
wherein the layer comprises a charged conductive material as defined and described herein,
wherein the conductive material is connected to a power supply and wherein the power supply and conductive material are arranged such that the power supply is capable of inducing a voltage across the layer, such that a capacitor structure is formed, and
such that water-containing elements are electrolysed by the capacitor structure upon contact with the antimicrobial and/or antiviral layer, thereby inactivating germs contained in the water-containing element.

In an embodiment, the germ is a virus, a bacterium, or a fungus. In an embodiment, the germ is a virus, for example a coronavirus such as Sars-CoV-2, a flu virus, HPV, HIV, or norovirus.

### FIGURES

FIG. 1: Water electrolysis caused by applying 4 volts between every two neighbouring (interdigitated) Ti/Au electrodes deposited on Si/SiO₂ in a water drop of about 200 microlitres.

FIG. 2: Top view of an example of an insulator substrate according to the invention, where the strips of conductive material are arranged into grids of strips, and where the grids are connected to the negative and positive poles of a power supply in an alternating fashion. The strips of conductive material are arranged to form an interdigitated electrode structure on a surface of the substrate.

FIG. 3: FIG. 3a is a cross-section illustration through a surface on which two-layer conductive electrodes are laid. FIG. 3b is a cross-section illustration through a surface including etched trenches in which conductive electrodes are deposited.

FIG. 4: A schematic illustrating an interdigitated array in accordance with the present invention with the array shown in exploded detail.

FIG. 5: Simulation results which depict electric potential and the electric field lines when a voltage difference of 4V is applied to the neighbouring 2 μm wide conducting lines on a glass surface.

FIG. 6: An image of a photomask which has been used to transfer an array as per the present invention onto a transparent substrate. The mask demonstrates the transparency of the array pattern.

FIG. 7: Cross sectional view of the strips of conducting material on an insulator substrate according to the invention. The protective layer and hygroscopic component are optional.

FIG. 8: Schematic of an example of an insulator substrate according to the invention. A row of solar units is used as the power supply.

FIG. 9: Schematic of an example of an insulator substrate according to the invention. A battery unit is used as the power supply.

FIG. 10: SEM images of silver nanowires before (A) and after (B) purification

FIG. 11: Photograph of blade coated ink electrodes at different blending ratios for transparency comparison; (A) Formulation of AgNWs in ethanol with PolyBioWire® ink at a blending ratio of 1.5:1. Blade coated ink electrodes show sheet resistances of 8-20Ω/square and transparency of 82.4% at 550 nm; (B) Formulation of AgNWs in ethanol with PolyBioWire® ink at a blending ratio of 1:1. Blade coated ink electrodes show sheet resistances of 5-8Ω/square and transparency of 56.7% at 550 nm; (C) PolyBioWire® ink. Blade coated ink electrodes show sheet resistances of 3-4Ω/square and transparency of 42. % at 550 nm.

FIG. 12: Surface of spike glycoprotein (S) structures in closed form of MHV and Sars-Cov-2 viruses and their charge values in mV.

FIG. 13: Design configuration used to test the virucidal properties of a weak electric field. On the lefthand side, the MHV virus is placed in between two aluminium electrodes connected to a power source. On the right-hand side, the MHV virus is placed on an interdigitated electrode structure printed on the surface of a PET film connected to a power source.

FIG. 14: Dilution and test plate configurations. Each treatment was tested 4 times and placed in quadruplicate in the test plate.

FIG. 15: Reed-Muench Calculator used in an excel sheet to calculate the TCID50/ml of each treatment.

FIG. 16: Log reduction in infectivity of MHV at 60 s (left) and 30 s (right) contact times.

FIG. 17: Graph showing the mean TDIC50/cm² values for human coronavirus NL63 following a contact time of 10 sec with test and reference control materials. Error bars are standard error of the mean.

FIG. 18: Graph showing the mean TDIC50/cm² values for human coronavirus NL63 following a contact time of 2 sec with test and reference control materials. Error bars are standard error of the mean.

FIG. 19: CAD design of electrodes suitable for smartphone and smartwatch antimicrobial/antiviral touch screens.

FIG. 20: Ti/Au interdigitated array on a tempered glass smartwatch screen protector, in which FIG. 20B is a closeup of the array, illustrating transparency.

FIG. 21: Microscope images of a Ti/Au interdigitated array after resist lift off process on a tempered glass smartwatch screen protector.

FIG. 22: Electrical stability testing of Ti/Au electrodes fabricated on a glass substrate. FIGS. 22A-C show a water droplet on the array at 0, 3.5 and 4 volts, respectively. FIG. 22D shows the increases in voltage applied to the array and FIG. 22E shows the stability of the current over time.

FIG. 23: Height profiling of interdigitated silver electrodes. The lefthand image demonstrates the location of the device where measurements were performed and a height profile graph is shown on the right.

FIG. 24: images of a transparent array showing the depth of micro-imprinted trenches before (FIG. 24A) and after (FIG. 24B) filling with silver ink and analysis of the resulting silver wires.

### DETAILED DESCRIPTION OF THE INVENTION

The present invention relates to a substrate having an antimicrobial and/or antiviral coating. The coating may be a film layer, said film layer comprising a charged conductive material.

1. Antimicrobial and/or Antiviral Substrate

In one aspect, the present invention relates to an insulator substrate having an antimicrobial and/or antiviral layer thereon, said layer comprising a charged conductive material connected to a power supply, wherein the power supply and the conductive material are arranged such that the power supply is capable of inducing a voltage across the layer, such that a capacitor structure is formed on a surface of the substrate.

The antimicrobial and/or antiviral properties of the substrate of the invention relies, inter alia, on electrolysing the water content that accompanies viruses, bacteria, and fungi upon contact with the surface of the substrate of the invention.

It is well known that applying a small amount of electricity to water breaks down the water's molecules, lowering its natural surface tension and creating positively and negatively charged ions. Applying a direct current (DC) voltage to the water produces ionised water that can change the pH of the water by the movement of ions. The water produced at the anode decreases in pH due to the increase of H⁺ ions, and the redox potential (ORP) increases, resulting in a strong oxidative state. The pH rises due to the increase of ions, resulting in a reducing state. In addition, strongly acidic electrolysed water, which contains hypochlorous acid (HOCl) as a major active ingredient, is produced at the anode of the electrode. Physicochemical analysis (ESR) has demonstrated that hydroxyl radical (·OH) and hydrogen peroxide (H₂O₂) generated in the strongly acidic electrolysed water, combined together, have a strong bactericidal/virucidal effect. Additionally, strongly alkaline electrolysed water can be produced as a by-product at the cathode.

To demonstrate this, an interdigitated Ti/Au electrode was fabricated onto a Si/SiO₂ substrate in accordance with the present invention. A thin layer (1 μm to 2 μm) of photosensitive polymer (Photoresist AZ5214E) was first spun on a silicon/Silicon dioxide (Si/SiO₂) wafer. To remove the solvents and solidify the photoresist, the wafer was baked at 110° C. for 2 minutes. To transfer the electrode pattern, a laser beam writer was used, which exposed the photoresist at 260 mJ/cm². The wafer was developed in an AZ351B developer for 40 seconds and then rinsed in DI water and dried using dry nitrogen gas. Having developed, Ti and Gold were evaporated using a thermal evaporator. Ti was the thickness of 20 nm, and the gold was 150 nm. However, the thickness of Ti can be from 1 nm to 50 nm and gold can be from 20 nm to 500 nm. The lift-off process, which involves soaking the wafer in acetone for a few hours, was then performed to remove Ti/Gold from unwanted areas. The resulting electrodes had a width of 8 μm and length of 3 cm. Spacing between the electrodes was 100 μm to give a theoretical transparency of 92%.

To test the electrolysis function of these interdigitated electrodes, the array was placed in a water drop of about 200 microliters and 4V was passed between every two neighbouring electrodes. As shown in FIG. 1, electrolysis was clearly observed and evidenced by the bubbles that were created during this process.

Ionised water, having an adjusted pH or redox potential, has bactericidal and antiviral effect. The DNA or RNA or protein of virus and bacteria are damaged when they are treated by strongly acidic electrolysed water. In addition, alkaline water that contains a trace amount of sodium hydroxide (NaOH) has emulsification properties to fat and oils and can dissolve proteins in viruses and bacteria.

A significant mode of transmission of infectious disease occurs when the droplets of body fluid, such as saliva, of an infected person containing virus, bacterium, or fungus is released due to coughing or sneezing and lands on surfaces. Viruses, bacteria, or fungi maintain a certain water content. This water, even in quantities as low as in the micromolar range, can become electrolysed upon contact with an insulator substrate having a layer comprising a charged conductive material and hygroscopic component (e.g. a hydrophilic polymer) thereon according to the present invention, thus inactivating the pathogen as a result.

The antimicrobial and/or antiviral properties of substrate of the invention also relies, inter alia, on the destabilisation of the viral or microbial nanoparticles, by creating an electric voltage. For example, viral particles typically have an isoelectric point (IEP) of 3.5-7, which means that, at these pH values, viral particles can maintain less colloidal stability explaining why they generally remain in an unstable zeta potential range. Upon contact with the surface of the substrate of the invention, viral particles are exposed to an electric field. This has the effect of changing the zeta potential of the viral particles, affecting their electrokinetic properties thus making them aggregated and unable to enter host cells.

In an embodiment, the conductive material is arranged into at least two strips, or a plurality of strips, on a surface of the substrate, wherein the strips are spaced apart from each other, and wherein the strips are alternately connected to positive and negative connections of the power supply, so as to induce a voltage across the layer between adjacent strips. In an embodiment, the strips of conductive material are arranged to form an interdigitated electrode structure on a surface of the substrate.

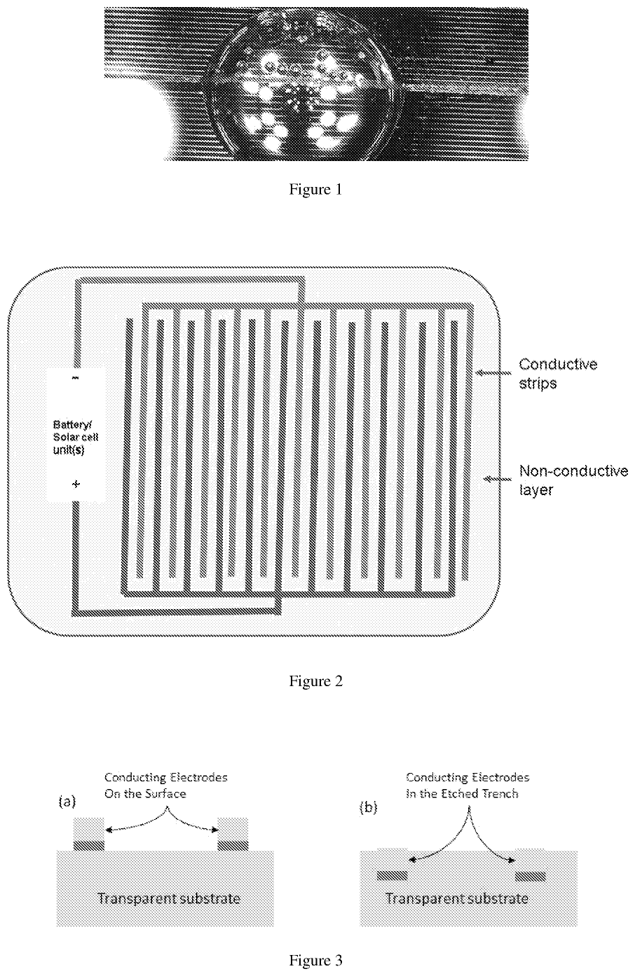

In an embodiment, the strips are parallel to each other. The strips may be arranged into grids of strips, wherein the grids are connected to the negative and positive poles of a power supply in an alternating fashion (FIG. 2). In an embodiment, a plurality of strips creates an interdigitated structure, wherein each alternative strip is connected to each other forming a first group, and each other alternative strip is connected to each other forming a second group. The first group is further connected to a positive pole of a power supply such as battery or a solar cell, and the second group is further connected to a negative pole of the power supply. An interdigitated geometry is simple to fabricate and may easily cover a whole surface. An example of such an arrangement or array is illustrated in FIG. 2 and, in more detail, in FIG. 3. However, multilayer and sandwich geometries are also encompassed by the present invention.

In an embodiment, the conductive material is arranged into at least two, or a plurality of, strips on a surface of the substrate, wherein the strips are spaced apart from each other, and wherein the strips are alternately a photocathode and a photoanode, so as to induce a voltage across the layer between adjacent strips. Photoactive conductive materials produce current or voltage under indoor and/or outdoor illumination.

In an embodiment, the voltage induced by the power supply is in the range of 10 mV to 50V. In another embodiment, the voltage induced by the power supply is in the range of 1V to 10V. In a particular embodiment, the voltage induced by the power supply is in the range of 1V to 5V. Ideally, a minimum voltage is 1.2V with a particularly effective voltage being between 1.3V and 4V. When a voltage is applied between the electrodes, an electric field is created.

In a non-touch state, no or a very little current of less than one micro-Ampere flows through the electrodes. However, if there is some or any humidity on the substrate surface from the atmosphere, a conducting path will be created between the electrodes and any airborne microbes/viruses that land on the substrate will be killed.

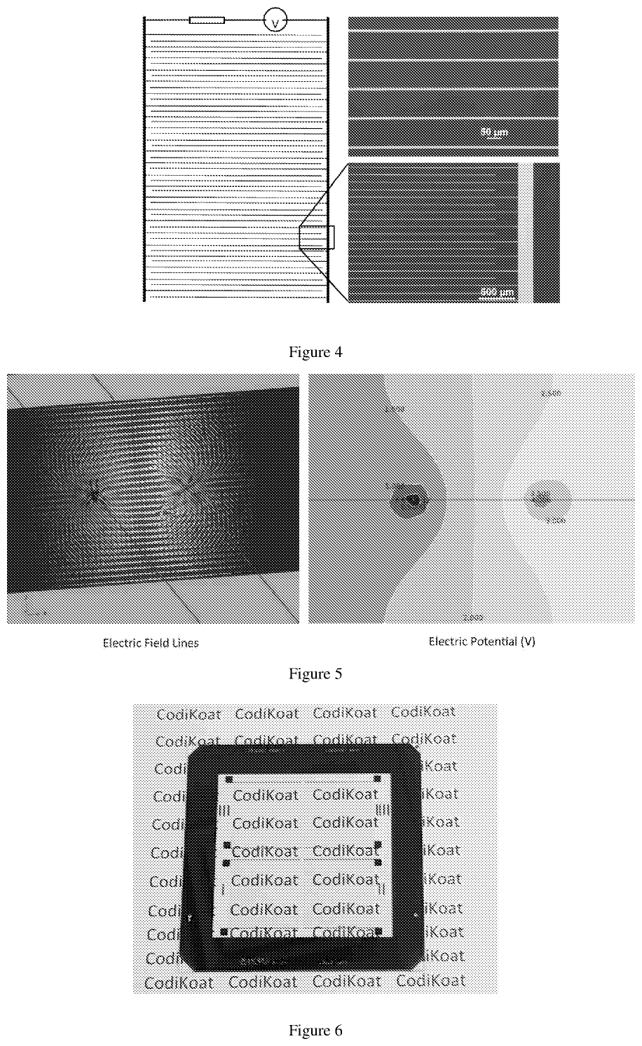

In the touch state, depending on the area of contact and the conductivity of the touching object such as skin, higher current flows between the electrodes because water, either from skin moisture or water from the microbe(s), electrolyses and creates electrons in the circuit. FIG. 4 shows the simulation results which depict electric potential and the electric field lines when a voltage difference of 4V is applied between neighbouring, interdigitated 2 μm wide electrodes separated by 100 μm on a glass surface. The potential difference between the electrodes causes water electrolysis.

In an embodiment, the conductive material comprises at least one metal, optionally selected from silver, aluminium, zinc, copper, titanium, chromium, iron, platinum, palladium, tungsten, and gold, together with mixtures, combinations and alloys thereof. In an embodiment, the conductive material comprises a metal oxide, optionally selected from nickel-, cobalt- and iron-based oxides, indium tin oxide and fluorinated tin oxide, as well as mixtures and combinations thereof. In an embodiment, the conductive material comprises a doped metal oxide, optionally wherein the doped metal oxide is aluminium doped zinc oxide. In an embodiment, the conductive material comprises a non-metal such as graphite, graphene or carbon, for example carbon nanostructures such as carbon nanotubes. It will be appreciated that the invention also encompasses combinations, mixtures, alloys, and multilayers.

In an embodiment, the conductive material consists of at least one metal, optionally selected from silver, aluminium, zinc, copper, titanium, iron, chromium, platinum, palladium, tungsten, and gold, as well as combinations, alloys, and multilayers thereof. In another embodiment, the conductive material consists of a metal oxide, optionally selected from nickel-, cobalt- and iron-based oxides, indium tin oxide and fluorinated tin oxide and mixtures and combinations thereof. In another embodiment, the conductive material consists of a doped metal oxide, optionally wherein the doped metal oxide is aluminium doped zinc oxide. In another embodiment, the conductive material consists of a non-metal such as graphite, graphene or carbon, for example carbon nanostructures such as carbon nanotubes, together with mixtures and combinations thereof.

In an embodiment, the conductive material comprises nanoparticles. In an embodiment, the conductive material comprises gold nanoparticles.

In a particular example, the conductive material may be formulated as an ink as described herein. Suitable metals for such formulation include gold, titanium, chromium, aluminium, and copper, and combinations and alloys thereof. These metals may be metal oxides. They may alternatively or in addition have a nanoparticulate form. The ink may also comprise a non-metal such as graphite, graphene or carbon, as well as mixtures and combinations of metals and non-metals.

Where photocathode and photoanode strips are present, the conductive material may comprise photoactive metal nanoparticles, metal oxide nanoparticles, metal sensitised with photoactive dyes, and/or metal oxides sensitised with photoactive dyes.

In an embodiment, the strips are made of a single layer or multilayers of conductive material. In an embodiment, the strips are made of a single layer of conductive material. In an embodiment, the strips are made of multilayers of conductive material. In an embodiment, the strips are made of one or more metal and/or conductive metal oxide. In an embodiment, the strips are made of a single layer of one or more metal and/or conductive metal oxide. In an embodiment, the strips are made of multilayers of one or more metal and/or conductive metal oxide. In an embodiment, the strips are made of or include a conductive non-metal such as graphite, graphene or carbon.

It will be appreciated that the array of strips may be applied either directly on a surface or applied on a polymer, glass, or sapphire transparent protector which is then applied to device surface.

It will also be appreciated that the array may be deposited directly onto a surface and so the conductive material, once applied, sits proud of the surface. Alternatively, the pattern of the array may be etched into the surface so the conductive material, once applied, sits within the surface and the upper, exposed surface of the conductive material is substantially flush with the surface in which the array is sited.

In an embodiment, the antimicrobial and/or antiviral layer is substantially transparent or semi-transparent. For example, the width of the electrodes may be 2 to 100 times smaller than the spacing between the electrodes. In this way, an overall transparency of 50% to 99% in the visible spectrum is achieved. For example, the width of the electrodes may be between about 1 μm and about 5 μm or between about 10 μm and 200 μm. Ideally, the thickness of the conducting electrodes is between 0.01 mm and 10 nm, is between about 20 nm and about 500 nm, is between about 1 μm and about 10 μm, or is less than 5 μm.

As illustrated in FIG. 6, it is possible to make the array of conductive material transparent. The figure shows the image of interdigitated electrodes patterned on a transparent glass substrate. The area inside the black boxes has interdigitated lines. Underneath the substrate is printing which can easily be read through the interdigitated electrodes. This is due to small electrode width and large electrode spacing which look transparent to the naked eye and demonstrates that the substrate of the present invention has good transparency of more than 90%.

In one example, the conductive material is made of indium tin oxide, fluorinated tin oxide, and aluminium doped zinc oxide. In an embodiment, the conductive material is indium tin oxide (a doped tin oxide) and the strips of conducting material have a thickness in the range of 5 nm-1000 μm. Preferably the thickness is 25 nm. Preferably, the thickness is 50 nm. Preferably the thickness is 100 nm. In another embodiment, the conductive material is fluorinated tin oxide and the strips of conducting material have a thickness in the range of 5 nm-1000 μm. Preferably the thickness is 25 nm. Preferably, the thickness is 50 nm. Preferably the thickness is 100 nm. In another embodiment, the conductive material is aluminium doped zinc oxide, and the strips of conducting material have a thickness in the range of 5 nm-1000 μm. Preferably the thickness is 25 nm. Preferably, the thickness is 50 nm. Preferably the thickness is 100 nm.

Alternatively, in an embodiment, the antimicrobial and/or antiviral layer is substantially opaque and either the conductive material and/or the substrate may include colour or pattern, such as a design to blend or match with surroundings in which the substrate is used or applied. Optionally, the conductive material is made of silver, aluminium, zinc, copper, titanium, iron, platinum, nickel, cobalt, palladium, or gold. In an embodiment, the conductive material is aluminium and the strips of conducting material have a thickness in the range of 5 nm-1000 μm. Preferably the thickness is 25 nm. Preferably, the thickness is 50 nm. Preferably the thickness is 100 nm.

In another embodiment, the conductive material is zinc and the strips of conducting material have a thickness in the range of 5 nm-1000 μm. Preferably the thickness is 25 nm. Preferably, the thickness is 50 nm. Preferably the thickness is 100 nm.

In another embodiment, the conductive material is copper and the strips of conducting material have a thickness in the range of 5 nm-1000 μm. Preferably the thickness is 25 nm. Preferably, the thickness is 50 nm. Preferably the thickness is 100 nm.

In another embodiment, the conductive material is titanium and the strips of conducting material have a thickness in the range of 5 nm-1000 μm. Preferably the thickness is 25 nm. Preferably, the thickness is 50 nm. Preferably the thickness is 100 nm.

In another embodiment, the conductive material is iron and the strips of conducting material have a thickness in the range of 5 nm-1000 μm. Preferably the thickness is 25 nm. Preferably, the thickness is 50 nm. Preferably the thickness is 100 nm.

In another embodiment, the conductive material is silver and the strips of conducting material have a thickness in the range of 5 nm-1000 μm. Preferably the thickness is 25 nm. Preferably, the thickness is 50 nm. Preferably the thickness is 100 nm.

In an embodiment, the distance between each adjacent strips of conducting material on the surface of the substrate independently is in the range of 0.5 μm to 1 cm, preferably in the range of 2 μm to 200 μm, more preferably in the range of 2 μm to 300 μm. In an embodiment, the width of each strip of conducting material independently is in the range of 0.5 μm to 1 cm, preferably in the range of 2 μm to 500 μm, more preferably in the range of 2 μm to 300 μm. In a particular example, the width of each strip of conducting material may be in a range of between about 1 μm and about 5 μm. In another example the width of each strip of conducting material may be in a range of between about 10 μm and about 200 μm.

In an embodiment, the thickness or depth of each strip of conducting material independently is in the range of 5 nm to 1000 μm, preferably in the range of 500 nm to 500 μm, more preferably in the range of 1 μm to 100 μm. In a particular example, the thickness or depth of each strip of conducting material may be in a range of between about 20 nm and about 500 nm. In another example the thickness or depth of each strip of conducting material may be in a range of between about 1 μm and about 10 μm.

The presence of water around the virus, bacterium, or fungi on the surface of the substrate of the invention can be increased by the addition of a hygroscopic component which is capable of absorbing and storing humidity from the environment. Advantageously, hygroscopic components are natural, non-toxic, inexpensive, and widely available. In an embodiment, the antimicrobial and/or antiviral film layer further comprises a hygroscopic component. Preferably, the hygroscopic component is selected from rock salt, silica sand, silica gel, CaCl₂, a hydrophilic polymer, or a glycosaminoglycan. In an embodiment, the hygroscopic component is a hygroscopic polymer. Preferably, the hygroscopic polymer is polyvinyl alcohol or polyethylene glycol. Preferably, the hygroscopic polymer is a mixture of polyvinyl alcohol and polyethylene glycol. In an embodiment, the hygroscopic agent is a glycosaminoglycan. Preferably the hygroscopic agent is a non-sulphated glycosaminoglycan, for example hyaluronic acid. In an embodiment, the hygroscopic component comprises an aqueous component, preferably water. Preferably, the hygroscopic component is mixed with a photo-crosslinking agent to form crosslinks between the hygroscopic component and the photo-crosslinking agent under thermal treatment or under exposure to light (e.g. UV light), thus preventing the hygroscopic component to become soluble under continuous exposure to water and/or humidity.

The presence of water around the virus, bacterium, or fungi on the surface of the substrate of the invention can also be increased by treating the surface of the insulator substrate beneath the antimicrobial and/or antiviral film layer with oxygen plasma or UV ozone, such that the surface tension of the surface of the insulator substrate is reduced. This will cause the landing body fluid droplet, such as saliva, to expand as much as possible between the strips of conducting material of the antimicrobial and/or antiviral film layer and hence maximise the possibility of making contact with the strips of conducting material. In an embodiment, the surface of the insulator substrate beneath the antimicrobial and/or antiviral film layer has been pre-treated with oxygen plasma or UV ozone, such that the surface tension of the surface of the insulator substrate is reduced. In an embodiment, the surface of the insulator substrate beneath the antimicrobial and/or antiviral film layer has been pre-treated with oxygen plasma. In an embodiment, the surface of the insulator substrate beneath the antimicrobial and/or antiviral film layer has been pre-treated with UV ozone.

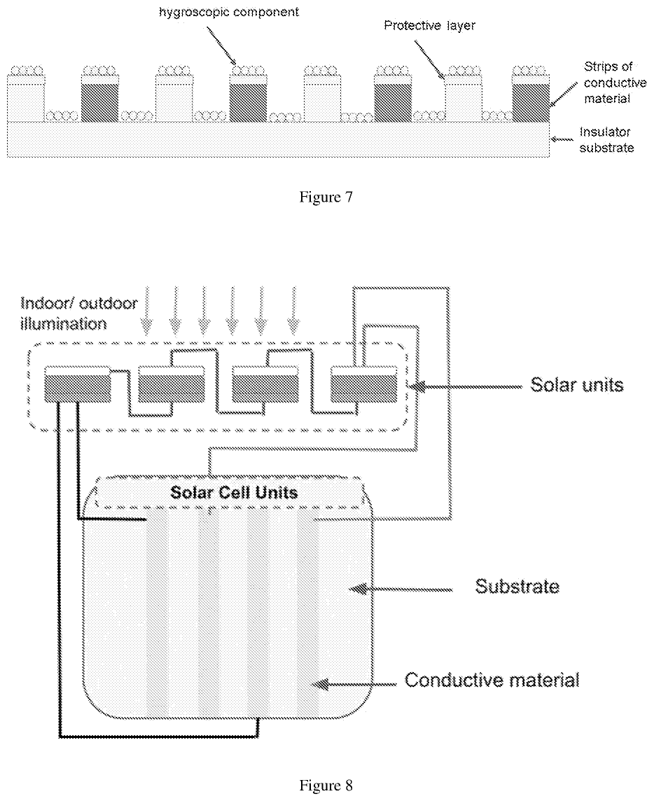

In an embodiment, the antimicrobial and/or antiviral layer is partially or fully covered by a protective layer (FIG. 7). Optionally, the protective layer is selected from epoxy or any non-conductive metal oxide such as aluminium oxide or silicon dioxide. The protective layer protects the strips of conductive material from degradation during exposure to humidity.

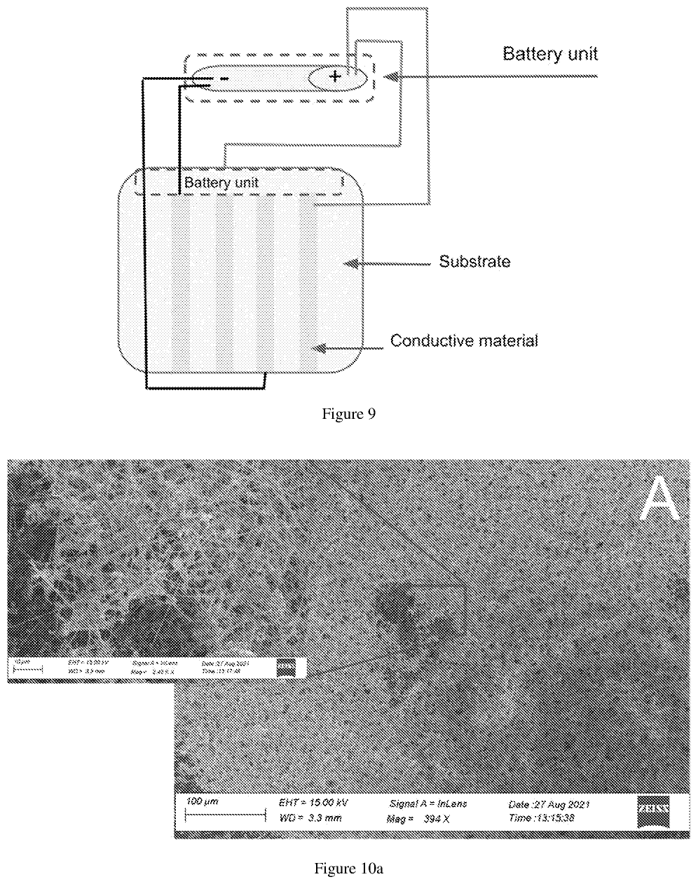

The electrode array may be powered either by a standalone battery or be powered by the battery of an electronic device, such as a mobile telephone or a tablet, to which the array is applied using appropriate connections such as USB or pogo pins. The connection to an internal battery of a device may be made through a USB power point used for charging or a similar dedicated power point on or within the device. Alternatively or in addition, the connection may be made on the surfaces of a device using designed connections. The power supply may be a solar cell or a battery (FIGS. 2, 8 and 9). In an embodiment, the power supply is coated and/or placed as close to the edges of the substrate surface as possible to create the highest antiviral area on the surface of the substrate. One or more surfaces of the substrate may be coated with a power supply. For example, a continuous row of solar cells and/or batteries is coated on all the surfaces of the substrate (FIG. 8).

In an embodiment, the power supply is a solar cell. Preferably, the solar cell is an organic or amorphous silicon solar cell, preferably wherein the solar cell is an OPV (organic photovoltaic), a DSSC (Dye-sensitised solar cells), a copper indium gallium selenide (CIGS) solar cell, or a flexible silicon solar cell. In an embodiment, the solar cell is commercially available. In another embodiment, the solar cell is a printable solar cell. In an embodiment, the solar cell is a dye sensitised solar cell composed of organic polymers (for example conjugated polymers such as thiophene-based polymers), small molecules (for example conjugated small molecules, fullerene based small molecules, non-fullerene based small molecules, perylene small molecules), and/or hybrid organic-inorganic structure arranged as photoactive materials.

In an embodiment, each solar cell unit has an area of 1 mm² to 100 cm². In an embodiment, each solar cell unit has an area of 1 mm² to 50 cm². In an embodiment, each solar cell unit has an area of 1 mm² to 10 cm². In an embodiment, each solar cell unit has an area of 1 mm² to 25 mm². Preferably, each solar cell unit has an area of 2.25 mm². Preferably, each solar cell unit has an area of 4 mm². Preferably, each solar cell unit has an area of 10 mm². Preferably, each solar cell unit has an area of 1 cm². In an embodiment, a surface of a substrate according to the invention has 2 to 10 solar cell units. Photoactive materials used in the solar cell have a thickness in the range of 100 nm to 2 μm, preferably in the range of 300 nm to 500 nm. The electron transport layer (ETL) has a thickness in the range of 25 nm to 500 nm, preferably in the range of 50 nm to 100 nm. The hole transport layer (HTL) has a thickness in the range of 25 nm to 500 nm, preferably in the range of 50 nm to 100 nm. In an embodiment, the cathode material is aluminium, silver, calcium and silver, calcium and aluminium, or lithium fluoride and aluminium, wherein the cathode has a thickness in the range of 50 nm to 500 nm, preferably in the range of 100 nm to 200 nm. In an embodiment, the cathode material is lithium fluoride, wherein the cathode has a thickness in the range 0.1 nm to 2 nm, preferably 1 nm. In an embodiment, the cathode material is calcium, wherein the cathode has a thickness in the range 2 nm to 50 nm, preferably in 10 nm, more preferably 20 nm. In an embodiment, the anode material is a transparent conductive oxide, wherein the anode has a thickness in the range of 50 nm to 500 nm, preferably in the range of 100 nm to 200 nm.

In another embodiment, the power supply is a battery. Preferably, the battery has a thickness in the range of 1 mm to 10 mm, or in the range of 10 μm to 100 μm. In an embodiment, the battery is commercially available. In another embodiment, battery is a printable battery. In an embodiment, the battery is a thin or ultra-thin battery having a voltage in the range of 10 mV to 50V. Preferably the battery has a voltage in the range of 1V to 5V. Preferably, the battery has a voltage of 1.5V. Preferably, the battery has a voltage of 2V. Preferably, the battery has a voltage of 2.5V.

In an embodiment, the conductive material is connected to the power supply by a wire. In an embodiment, the wire comprises: a metal, a conductive metal oxide, a doped metal oxide, and/or graphene or carbon, for example carbon nanostructures such as carbon nanotubes, as defined and described herein. In an embodiment, the wire is made of the same material as the material used to make the strips of conductive material. In another embodiment, the wire is made of a material different from the material used to make the strips of conductive material.

In an embodiment, the insulator substrate is made of an insulator material. In another embodiment, the insulator substrate is made of a poor semi-conductor material, optionally selected from conjugated polymers, small molecules, and/or metal oxides.

In an embodiment, the insulator substrate is flexible, optionally wherein the insulator substrate is a polymeric adhesive film, fabric, plastic, paper, fibres, or amorphous silicon. In another embodiment, the insulator substrate is non-flexible, optionally wherein the insulator substrate is glass, plastic or ceramic. In one example, the conductive array may be applied directly onto a surface or applied on a polymer, glass or sapphire transparent protector which is then be applied to the surface. The surface may be a mobile telephone or tablet.

The present invention can be integrated into manufacturing of a wide range of products, in particular on frequently touched surfaces. The present invention produces a fast-acting and long-lasting antimicrobial and/or antiviral shield which inactivates viruses and bacteria on a wide range of surfaces and last the lifetime of the product. As such, the present invention also relates to an article of manufacture comprising the insulator substrate defined herein. In an embodiment, the article of manufacture is selected from an electronic device such as a tablet or mobile phone, a touchscreen, a keypad, a keyboard, a button, a handle, a screen protector, a piece of furniture such as a seat, a table, an arm rest, a toilet seat, a cup, a worktop, and an appliance, such as a kitchen appliance, a door plate, a water tap, a car steering wheel, a car key, food packaging, stationary, and a bank note.

Where the article of manufacture is an electronic device, the original power supply of the electronic device may be used as the power source.

### 2. Method of Manufacture

The invention further relates to the use of printing, preferably inkjet printing, in the manufacture of an antimicrobial and/or antiviral layer comprising a charged conductive material as defined herein on a surface of an insulator substrate, or of an antimicrobial and/or antiviral film layer comprising a charged conductive material as defined herein on a surface of an article of manufacture as defined herein. The invention also relates to the use of atomic layer deposition (ALD) in the manufacture of an antimicrobial and/or antiviral layer comprising a charged conductive material as defined herein on a surface of an insulator substrate, or of an antimicrobial and/or antiviral layer comprising a charged conductive material as defined herein on a surface of an article of manufacture as defined herein.

The invention also relates to the use of lithography in the manufacture of an antimicrobial and/or antiviral layer comprising a charged conductive material as defined herein on a surface of an insulator substrate, or of an antimicrobial and/or antiviral layer comprising a charged conductive material as defined herein on a surface of an article of manufacture as defined herein. The term “lithography” encompasses photolithography, nanoimprint lithography, e-beam lithography, laser lithography, laser etching, projection lithography. ion beam lithography and x-ray lithography.

In one example, electrodes may be either fabricated by depositing conducting electrode material after lithography and lift-off process or by depositing conducting electrodes before lithography and following by the etching process.

In one embodiment, interdigitated electrodes may be fabricated on a surface by first transferring a pattern, using a lithography process as defined herein, in a polymer layer called a resist and subsequently etching and then depositing conducting (metal) electrodes and multilayer combinations and alloys thereof using a lift-off process. In a particular example, the geometry of the electrode array is such that, after lithography, the surface is plasma or wet etched and the conducting electrodes are deposited in trenches so that a smoother surface is achieved in the final device. As an example, FIG. 3a illustrates surface in cross-section with electrodes fabricated on the surface. In another example, conductive electrodes may be deposited in trenches which have been created after etching the transparent substrate. As can be seen in FIG. 3b, etched trenches ideally have nearly the same depth as the total thickness of the conducting electrodes. Trenches may be created using either a wet or a dry etching processes. Ideally, etching is carried out using the same lithography step that is used for depositing the electrodes.

In a particular embodiment, the substrate of the invention includes interdigitated electrodes, a current regulated circuit, and a power source. FIG. 4 shows a particular design for an interdigitated array in accordance with the present invention. Antiviral and antibacterial activity requires the application of a small voltage difference, that may vary from 1V to 10V, between the interdigitated conducting electrodes. It will be appreciated that the current regulated circuit may be a constant resistor or variable resistor controlled by an IC circuit and a processor. The role of a current regulated circuit is to limit current flow to protect the power source and the substrate as a whole in a situation where there is a short-circuiting, or an excessive amount of water is present on the surface such as during cleaning or spillage.

It will be appreciated that the (interdigitated) electrodes described herein may be arranged in different forms. In the figures, widths, lengths, thicknesses, and the like of elements may be exaggerated for convenience and illustrative purposes. The final circuit design may be a combination of many parallel and series combinations of the electrodes, preferably interdigitated electrodes.

The invention also relates to the use of laser ablation or cutting system in the manufacture of an antimicrobial and/or antiviral layer comprising a charged conductive material as defined herein on a surface of an insulator substrate, or of an antimicrobial and/or antiviral layer comprising a charged conductive material as defined herein on a surface of an article of manufacture as defined herein, wherein laser ablation or cutting system is used to remove strips from a commercially existing conductive film to create strips of conducting material. Grids of conductive material can be made by laser cutting on a PET/plastic substrate coated with a transparent or non-transparent material (e.g. ITO, Al). The laser cut grids can then be adhered on an insulator substrate using adhesive.

It will be appreciated that thermal evaporation, electron beam evaporation, physical vapor deposition, chemical vapor deposition, sputter deposition, electroplating, inkjet printing, screen printing, flexographic printing, spray coating, roll to roll printing, 3D printing, nano/micro imprinting and atomic layer deposition, pulsed laser deposition, electroplating, electroless plating and sol gel are all suitable methods for manufacturing the antimicrobial and/or antiviral layer of the present invention.

The present invention further relates to a method of making an insulator substrate as defined herein or an article of manufacture as defined herein.

Inkjet printing can be used to fabricate a structure by depositing thin material layers from precursor inks in prescribed locations and in specific patterns by precision layering. Inkjet printing significantly reduces the generation of hazardous and environmentally sensitive waste and the expense associated with waste treatment. Therefore, the present invention also relates to a method of making a substrate having an antimicrobial and/or antiviral film layer thereon, said film layer comprising a conductive material, wherein the method comprises the steps of:
a) providing a precursor substrate and a conductive material ink, wherein the precursor substrate is made of an insulator material,
b) printing the conductive material ink on a surface of the precursor substrate.

In an embodiment, the insulator material is a poor semi-conductor material, optionally selected from conjugated polymers or small molecules.

In an embodiment, the conductive material ink comprises a metal, a metal oxide, and/or a doped metal oxide, and a solvent. Where a conductive material comprises a metal, the additional use of a metal oxide may increase the stability of the metal compound. Optionally, the metal and/or metal oxide is selected from silver, nickel-, indium tin oxide, fluorinated tin oxide, aluminium doped zinc oxide, aluminium, zinc, copper, chromium, titanium, iron, and gold. In an embodiment, the conductive material ink comprises graphite, graphene or carbon, for example carbon nanostructures such as carbon nanotubes, and a solvent. In an embodiment, the conductive material ink comprises nanoparticles. In an embodiment, the conductive material ink comprises gold nanoparticles. In an embodiment, the conductive material ink comprises photoactive metal nanoparticles, metal oxide nanoparticles, metal sensitised with photoactive dyes, and/or metal oxides sensitised with photoactive dyes, and a solvent. In an embodiment, the conductive material ink comprises photoactive metal nanoparticles and/or metal oxide nanoparticles. In an embodiment, the conductive material ink comprises metal sensitised with photoactive dyes, and/or metal oxides sensitised with photoactive dyes. In an embodiment, the solvent is ethanol.

In an embodiment, step b) of the method further comprises printing the conductive material ink to form at least two, or a plurality of, strips of conductive material on a surface of the precursor substrate, wherein the strips are spaced apart from each other. In an embodiment, step b) of the method is repeated to form multiple layers of conductive material.

The present invention also relates to a method of making a substrate having an antimicrobial and/or antiviral layer thereon, said layer comprising a conductive material, wherein the method uses atomic layer deposition (ALD), physical vapour deposition (PVD) or thermal evaporation. Thermal evaporation is a method of physical vapour deposition. In this process, a resistive heating source is used to evaporate solid materials in vacuum condition to form a thin film, ranging from just a few nanometres to hundreds of nanometres. The pressure for the evaporation is in the range of 10⁻⁶ mbar.

The present invention also relates to a method of making a substrate having an antimicrobial and/or antiviral layer thereon, said layer comprising a conductive material as defined and described herein, wherein the method uses a sputtering technique. According to this method, a desired source material is ion-bombarded at high vacuum conditions (in the range of 10⁻⁵ mbar) to produce a vapour of the source material, and to further produce a film of the material on a substrate. The gases used to generate plasma for ion bombardment are argon and/or nitrogen. The thickness of the film formed can vary in the range of nm to 1.1M.

In an embodiment, a photolithography process is used to produce a photomask (also called an optical mask) of the required pattern before printing the conductive material ink on the surface of the precursor substrate. This process helps in situations where the printing or deposition system may not be able to produce the pattern with the required resolution.

In an embodiment, step a) of the method further comprises treating the precursor substrate with oxygen plasma or UV ozone, such that the surface tension of the surface of the insulator substrate is reduced.

The present invention also relates to a method of making a substrate having an antimicrobial and/or antiviral layer thereon, said layer comprising a conductive material, wherein the method comprises the steps of:
a) providing a precursor substrate made of an insulator material and a substrate coated with a layer of conductive material,
b) removing strips of conductive material using laser ablation or cutting system, thereby forming strips of conducting material on the surface of the substrate, wherein the strips are spaced apart from each other.
c) adhering the laser-cut strips of conductive material on the precursor substrate made of an insulator material.

In an embodiment, the substrate coated with a layer of conductive material is an aluminium tape.

In an embodiment, the distance between each adjacent strips on the surface of the precursor substrate independently may be in the range of 0.5 μm to 1 cm, preferably in the range of 2 μm to 200 μm, more preferably in the range of 2 μm to 300 μm. In an embodiment, the width of each strip independently is about 2 to 100 times smaller than the distance between the strips. For example, the width may be in the range of 0.5 μm to 1 cm, preferably in the range of 2 μm to 500 μm, more preferably in the range of 2 μm to 300 μm. In an embodiment, the width of each strip may be between about 0.01 mm and 10 nm, preferably less than 5 μm. In a particular example, the width of each strip may be in a range of between about 1 μm and about 5 μm. In another example, the width of each strip may be in a range of between about 10 μm and about 200 μm.

In an embodiment, the thickness of each strip independently is in the range of 5 nm to 1000 μm, preferably in the range of 500 nm to 500 μm, more preferably in the range of 1 μm to 100 μm. In a particular example, the thickness of each strip may be in a range of between about 20 nm and about 500 nm. In another example, the width of each strip may be in a range of between about 1 μm and about 10 μm.

In an embodiment, the method described herein further comprises printing or placing a power supply on a surface of the precursor substrate, wherein the power supply and the conductive material are arranged such that the power supply is capable of inducing a voltage across the antimicrobial and/or antiviral layer on a surface of the precursor substrate. In an embodiment, the power supply is coated and/or placed as close to the edges of the substrate surface as possible to create the highest antiviral area on the surface of the substrate. One or more surfaces of the substrate may be coated with a power supply. For example, a continuous row of solar cells and/or batteries is coated on all the surfaces of the substrate (FIG. 8). In an embodiment, the power supply is a solar cell or a battery.

In an embodiment, the power supply is a solar cell, optionally wherein the solar cell is an organic or amorphous silicon solar cell, preferably wherein the solar cell is an OPV (organic photovoltaic), a DSSC (Dye-sensitised solar cells), a copper indium gallium selenide (CIGS) solar cell, or a flexible silicon solar cell. In an embodiment, each solar cell unit has an area of 1 mm² to 100 cm². In an embodiment, each solar cell unit has an area of 1 mm² to 50 cm². In an embodiment, each solar cell unit has an area of 1 mm² to 10 cm². In an embodiment, each solar cell unit has an area of 1 mm² to 25 mm². Preferably, each solar cell unit has an area of 2.25 mm². Preferably, each solar cell unit has an area of 4 mm². Preferably, each solar cell unit has an area of 10 mm². Preferably, each solar cell unit has an area of 1 cm². In an embodiment, a surface of a substrate according to the invention has 2 to 10 solar cell units. Photoactive materials used in the solar cell have a thickness in the range of 100 nm to 2 μm, preferably in the range of 300 nm to 500 nm. The electron transport layer (ETL) has a thickness in the range of 25 nm to 500 nm, preferably in the range of 50 nm to 100 nm. The hole transport layer (HTL) has a thickness in the range of 25 nm to 500 nm, preferably in the range of 50 nm to 100 nm. In an embodiment, the cathode material is aluminium, silver, calcium and silver, calcium and aluminium, or lithium fluoride and aluminium, wherein the cathode has a thickness in the range of 50 nm to 500 nm, preferably in the range of 100 nm to 200 nm. In an embodiment, the cathode material is lithium fluoride, wherein the cathode has a thickness in the range 0.1 nm to 2 nm, preferably 1 nm. In an embodiment, the cathode material is calcium, wherein the cathode has a thickness in the range 2 nm to 50 nm, preferably in 10 nm, more preferably 20 nm. In an embodiment, the anode material is a transparent conductive oxide, wherein the anode has a thickness in the range of 50 nm to 500 nm, preferably in the range of 100 nm to 200 nm.

In another embodiment, the power supply is a battery, optionally wherein the battery has a thickness in the range of 10 μm to 100 μm. In an embodiment, the battery is commercially available. In another embodiment, battery is a printable battery. In an embodiment, the battery is a thin or ultra-thin battery having a voltage in the range of 10 mV to 50V. Preferably the battery has a voltage in the range of 1V to 5V. Preferably, the battery has a voltage of 1.5V. Preferably, the battery has a voltage of 2V. Preferably, the battery has a voltage of 2.5V.

In an embodiment, the method described herein further comprises printing a wire, wherein the wire connects each strip of conductive material to the power supply. In an embodiment, the wire comprises: a metal, a conductive metal oxide, a doped metal oxide, and/or graphene or carbon, for example carbon nanostructures such as carbon nanotubes.

In an embodiment, the method described herein further comprises layering a hygroscopic component on the precursor substrate. In an embodiment, the hygroscopic component is selected from rock salt, silica sand, silica gel, CaCl₂, a hydrophilic polymer, or a glycosaminoglycan. In an embodiment, the hygroscopic component is a hygroscopic polymer. Preferably, the hygroscopic polymer is polyvinyl alcohol or polyethylene glycol. Preferably, the hygroscopic polymer is a mixture of polyvinyl alcohol and polyethylene glycol. In an embodiment, the hygroscopic agent is a glycosaminoglycan. Preferably the hygroscopic agent is a non-sulphated glycosaminoglycan, for example hyaluronic acid. In an embodiment, the hygroscopic component comprises an aqueous component, preferably water. The hygroscopic component can be deposited by making a hygroscopic powder directly on the surface of the insulator substrate. In particular, a hygroscopic component in a form of powder can be sprayed via an electrostatic spray gun. The sprayed micro/nanoparticles are formed on the surface of the insulator substrate, thus forming a medium between strips of conductive material. Alternatively, the hygroscopic component can be deposited by making thin films of hygroscopic component on the surface of the insulator substrate. In particular, a solution of a hygroscopic component is sprayed or casted on the surface of the insulator substrate to form a thin film. Strips of conducting material are then formed on the surface of this thin film via introduced coating techniques for metal strips.

In an embodiment, the method described herein further comprises depositing a partial or full protective layer on the conductive material. In an embodiment, the protective layer is deposited by physical vapour deposition, chemical deposition such as ALD, or printing. In an embodiment, the protective layer is deposited by physical vapour deposition. In an embodiment, the protective layer is deposited by ALD. In an embodiment, the protective layer is deposited by printing. In an embodiment, the protective layer is selected from epoxy or any non-conductive metal oxide such as aluminium oxide or silicon dioxide.

In an embodiment, the antimicrobial and/or antiviral layer is substantially transparent or semi-transparent, optionally wherein the antimicrobial and/or antiviral layer is made of nickel-, cobalt- and iron-based oxides, indium tin oxide, fluorinated tin oxide, aluminium doped zinc oxide and mixtures and combinations thereof. In another embodiment, the antimicrobial and/or antiviral layer is substantially opaque, optionally wherein the antimicrobial and/or antiviral layer is made of silver, aluminium, zinc, copper, titanium, iron, or gold.

In an embodiment, the substrate precursor is flexible, optionally wherein the substrate precursor is a polymeric adhesive film, fabric, plastic, paper, fibres, or amorphous silicon. In another embodiment, the substrate precursor is non-flexible, optionally wherein the substrate precursor is glass, plastic or ceramic.

The invention also relates to a method of depositing an insulator substrate as defined herein or an article of manufacture as defined herein.

### 3. Uses

The insulator substrate having an antimicrobial and/or antiviral layer thereon, said layer comprising a charged conductive material according to the present invention exhibit an antimicrobial and/or antiviral activity. In particular, the present insulator substrate produces a rapid (within seconds) inactivation of viruses, including coronavirus. This is achieved, at least in part, by using electricity and water electrolysis to provide rapidly deployable, fast-acting and long-lasting protection. This technology is capable of inactivating viruses such as coronavirus in seconds.

Standard tests to measure antiviral and antimicrobial properties include cytopathic effect (CPE) inhibition assay, plaque assay, qPCR assay, flow cytometry, and TCID50 infectivity assay.

A cytopathic effect (CPE) inhibition assay can be used to measure the antiviral property of the insulator substrate of the invention. Many combinations of cells and viruses can be used to measure viral infectivity via CPE assay. MTT (3-[4,5-dimethylthiazole-2-yl]-2,5-diphenyltetrazolium bromide) and ATP luminescence assays are both efficient and simple ways of quantifying the CPE. A range of viruses and host cell lines are used to quantify the cytopathic effect of a particular virus to a particular cell line after exposure to the coated substrates of the invention.

In summary the cells are seeded in cell culture plates and incubated for 18 to 24 hours. Aqueous suspensions of viral particles are then placed on a small size of the coated substrate material (e.g. 2 cm×2 cm sample) and also uncoated substrate (uncoated control) for a specified contact time (i.e. this is the time under investigation which specifies the speed of action of the coated substrate). The viral particles are then recovered from the substrate by rinsing serum-free medium on the substrate and the efficiency of this viral recovery is quantified using nanoparticle tracking analysis. The recovered viruses are used to infect the seeded host cells while using parallel sets of cells infected with untreated viruses as positive control and uninfected cells as negative control. Cell viability test of these cell sets is then carried out using the MTT assay kit which is purchased from a manufacturer (MTT Assay Kit (Cell Proliferation) (ab211091)) or ATP luminescence (commercial kit ViralToxGlo™ (Promega)).

A fluorescent cell viability assay can be used to measure the antimicrobial properties of the insulator substrate of the invention. To measure anti-microbial activity, fluorescent dyes are used throughout different techniques as a rapid way to distinguish between populations and determine the viability of cells. An example is the bacterial viability assay kit from Abcam (ab189818), which utilises two highly specific, ultrasensitive fluorescent reagents to quickly and easily assess the percentage of live and dead cells within a bacterial culture. The total cell stain is permeant to all cells and thus all bacteria within the culture will be stained allowing the total number to be calculated. The dead cell stain is impermeant to living cells and as such will only be able to enter and stain dead cells; this allows the number of dead cells to be calculated. The ratio of dead to live cells can then be quickly and easily calculated.

In this method, an aqueous suspension of the bacterial cells is placed on a small size of the coated substrate material (e.g. 2 cm×2 cm sample) and also uncoated substrate (uncoated control) for a specified contact time (i.e. this is the time under investigation which specifies the speed of action of the coated substrate). The suspension is then recovered from the substrate by rinsing serum free medium on the substrate. Cells are then harvested by spinning at 10,000 g for 10 min and resuspended in a buffer. Cells are incubated for 1 hour and spun at 10,000 g for 10 min. Cells are resuspended and the procedure is repeated. Total cell stain and dead cell stain are then added. Cells are either incubated for 1 hour and analysed with a microplate reader or incubated for 15 min and analysed with fluorescence microscope.

In one aspect, the invention relates to the use of the insulator substrate as defined herein, or the article of manufacture as defined herein, as an antimicrobial agent. The term “antimicrobial” encompasses bacteria and fungi.

The invention also relates to the use of the insulator substrate as defined herein, or the article of manufacture as defined herein, as an antiviral agent. In an embodiment, the germ is a virus, a bacterium, or a fungus. In an embodiment, the germ is a virus, for example a coronavirus such as Sars-CoV-2, a flu virus, HPV, HIV, or norovirus.

The invention also relates to the use of the insulator substrate as defined herein, or the article of manufacture as defined herein, for inactivating a germ.

The invention also relates to the use of an antimicrobial and/or antiviral layer for inactivating a germ,
wherein the layer comprises a charged conductive material as defined and described herein,
wherein the conductive material is connected to a power supply and wherein the power supply and conductive material are arranged such that the power supply is capable of inducing a voltage across the film layer, such that a capacitor structure is formed, and
such that water-containing elements are electrolysed by the capacitor structure upon contact with the antimicrobial and/or antiviral layer, thereby inactivating germs contained in the water-containing element.

In an embodiment, the germ is a virus, a bacterium, or a fungus. In an embodiment, the germ is a virus, for example a coronavirus such as Sars-CoV-2, a flu virus, HPV, HIV, or norovirus.

In one embodiment, inactivation of the germs occurs within 1 second to 10 minutes. Preferably, inactivation of the germs occurs within 1 to 60 seconds. More preferably, inactivation of the germs occurs within 30 seconds. More preferably, inactivation of the germs occurs within 15 seconds. More preferably, inactivation of the germs occurs within 5 seconds.

### EXAMPLES

### Example 1—Printing Indium Tin Oxide (ITO)

Commercial indium-tin-oxide (ITO) nanoparticles with D50=25 nm (Advanced Nano Products) were utilized in ITO ink formulation. The solid content of ITO nanoparticles in the ink was maintained at 15 wt % in the entire experiment. Ethanol was selected as a solvent for ITO ink. ITO nanoparticles were dispersed with a proper dispersant in the solvent by the 72 h ball-mill process and high speed mixing at 2000 rpm was carried out for 8 min followed by ultrasonic homogenizing process for 10 min. The formulated ITO ink was filtered through a 6 m polypropylene mesh in order to get rid of agglomerated ITO particles in the ink. The substrates were cleaned by acetone, ethanol and boiled isopropanol. UJ 200 inkjet-printing unit (Unijet) was used in the entire experiment, which is equipped with a piezoelectric nozzle with 19 m orifice from SEMJET (Samsung). The printing frequency, substrate temperature and pitch between ink drops were modulated in order to print uniform ITO films on the substrates. The volume of ink droplet was fixed at 30 pl throughout the entire experiment. The ITO film with the dimension of 20 mm×20 mm was printed on the glass substrate (25 mm×25 mm). The inkjet-printed TCO films were annealed in air atmosphere under microwave irradiation (2.45 GHz) with the maximum output power of 2 kW (Unicera; UMF-01). The thickness of the inkjet-printed ITO films was measured by Veeco Dektak 150 surface profiler and field emission scanning electron microscope (FE-SEM; Nova 200).

### Example 2—Printing Silver (Ag)

A commercial Ag ink (Dycotec Materials, with the high electrical conductivity (<6 mΩ/sqr/25 μm) at 100-140° C.) was used to print Ag-grid patterns by inkjet. Based on our experiment, the resistivity of the inkjet-printed Ag was less than 10 micro ohm-cm with the annealing temperature of 200° C.

### Example 3—Formulating and Printing with Graphene-Based Ink

Materials:

Natural graphite powder (Timrex® SFG15) was obtained from IMERYS Graphite & Carbon Co., Switzerland and Sodium-salt carboxymethyl cellulose (Na-CMC) (MW 700K) was purchased from Sigma-Aldrich. Timrex® SFG15 was selected because the graphite particle size is up to 15-20 μm which is suitable for flow in microchannels of microfluidiser.

Microfluidisation of Graphite and Formulation of Graphene Based Conductive Ink:

Microfluidisation is a homogenisation technique where a high pressure (up to 207 MPa) is applied to a liquid, forcing it to pass through a microchannel (diameter, d<100 μm). A key advantage over sonication and shear-mixing is that high γ up to 108 S-1 is applied to the whole liquid volume, not just locally.

Timrex® SFG15 graphite flakes were used as a starting material. Na-CMC was first mixed in deionised (DI) water and the flakes were then added and treated with a microfluidic processor with a Z-type geometry interaction chamber (LM20, Microfluidics Corp. USA). Mixtures were processed at the maximum pressure for this system (20,000 Psi). Graphite and Na-CMC mixtures with graphite C of 80 g·L−1 and Na-CMC C of 1 g·L⁻¹ in DI water were processed over 50 cycles. One cycle is defined as a complete pass through the interaction chamber. First cycles were with 200 μm channel installed (40 cycles with inlet pressure of 5000, 10000, 15000, and 20000 psi), and then the diameter of the channel was reduced to 100 μm (10 cycles with inlet pressure of 20000 psi)

Production of Mono-Bi-Few-Layer Graphene by Microfluidisation:

The resulting shear rate in the liquid γ was up to 108 s⁻¹, which is expected to lead to exfoliation of mono-bi-few-layers graphene (MBFLG) from the starting graphite material. A feed mixture of graphite (Timrex® SFG15) with a concentration of 20 g/L in ethanol, which was kept relatively low to prevent blockage of the channel in the microfluidiser, was run out. First cycles were with 200 μm channel installed (40 cycles with inlet pressure of 5000, 10000, 15000, and 20000 psi), and then the diameter of the channel was reduced to 100 μm (10 cycles with inlet pressure of 20000 psi). In order to remove unexfoliated graphite material, samples were centrifuged (1000 rpm to 10000 rpm at 10 min centrifugation time), retaining the supernatant. The supernatant was then recovered to be recognised as suspended MBFLG in ethanol.

Printable Inks Formulation:

Following microfluidisation, the dispersion was stabilised and dried at room temperature for 144 h. The inks were then deposited on a glass or polymer substrate using blade coating or screen-printing techniques. During printing/coating, shear was applied to the ink, to decrease viscosity, making the ink easier to print or coat. Additional information on the ink rheological behaviour and microstructure was obtained by well-known and standard oscillatory rheology measurements. For simplicity, blade coating was used to compare ink formulations. Inks were blade coated onto glass microscope slides (25×75 mm) using a polyimide (Kapton®) tape with different thickness to control the coated electrode thickness. Without any heating treatment or any further processing, the coated electrode films showed sheet resistances (Rs) of ˜9-11Ω/Square.

### Example 4—Printing of Wire

Conductive material can be printed by an ink-jet printer onto a surface of a substrate in the same way and using the same material as the strips of conductive material. For example, the print head can be mounted onto a computer-controlled three-axis gantry system and printing can be performed with applying varying voltages, lasting time, head frequency, temperature and inter-spacing between droplets to find the most optimised parameters for a specific substrate. Once the optimised parameters are found, the printing process can be performed using those parameters.

### Example 5—Deposition of a Hygroscopic Component

The Hygroscopic Component can be Deposited According to the Following Protocols:

Making a hygroscopic powder directly on the surface of the insulator substrate: A hygroscopic component in a form of powder can be sprayed via an electrostatic spray gun. The sprayed micro/nanoparticles are formed on the surface of the insulator substrate. This forms a medium between two metal strips.

Making thin films of hygroscopic component on the surface of the insulator substrate having strips of conductive material on its surface: A solution of a hygroscopic component is sprayed on the surface of the insulator substrate having strips of conductive material on said surface to form a thin film. Where the hygroscopic component is a hydrophilic polymer, the hydrophilic polymer is coated on top of the strips of conductive material as well as between the strips of conductive material.

### Example 6—Silver Nanowires Synthesis and their Transparent Conductive Ink Formulation

Materials:

Silver nitrate (99.8%), ethanol (99%), ethylene glycol (99.5%), polyvinylpyrrolidone (Mw≈40,000, Mw≈55,000), copper (II) chloride (CuCl₂) were purchased from Sigma-Aldrich, UK.

Silver nitrate (99.8%), ethanol (99%), Ethylene glycol (99.5%), Polyvinylpyrrolidone (Mw≈40,000, Mw≈

Silver Nanowires (AgNWs) Synthesis:

100 mL of Ethylene Glycol in a glass flask was heated at 150° C. for 1 h under continuous mechanical stirring. Reagent solutions of 0.004M Copper (II) Chloride, 0.2 M Poly(Vinyl Pyrrolidone) (PVP, MW: 55000), and 0.1 M of Silver Nitrate were prepared. After preheating, 800 μL of CuCl₂ solution was added, and the solution was heated for fifteen minutes. Finally, 30 mL of PVP solution and 30 mL of AgNO₃ solution was injected into the flask. The reaction flasks were purged with nitrogen gas during the preheating and reaction periods (160° C., 1 h). After nanowire formation, the reaction flask was cooled to room temperature.

Purification of Silver Nanowires by Selective Precipitation:

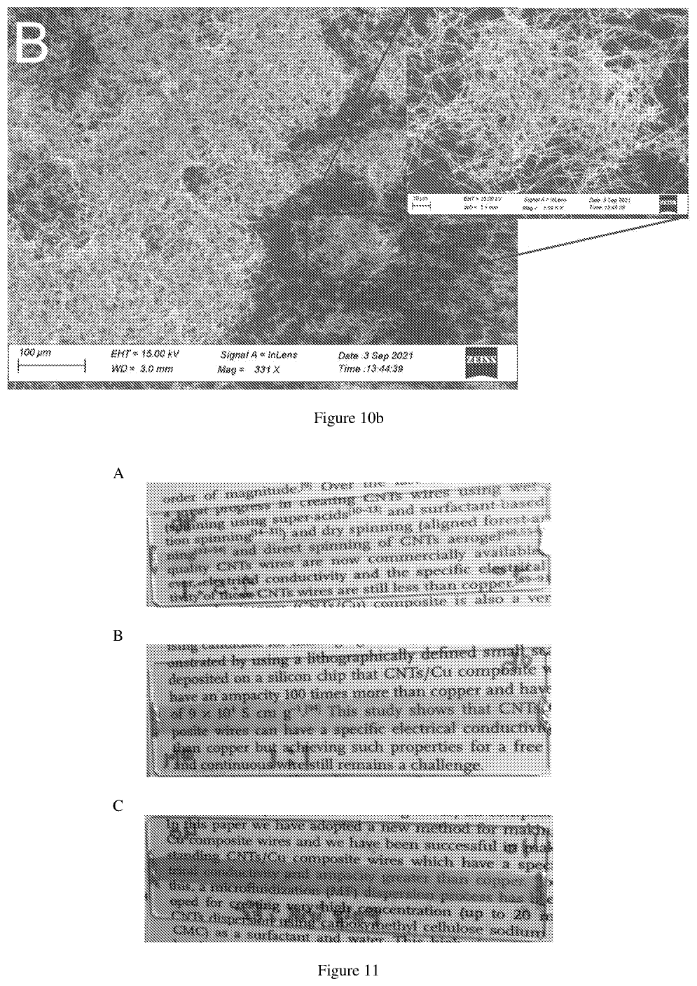

The as-synthesised AgNWs were diluted with ethanol and then centrifuged at 300-100 rpm for 1-6 min. After centrifugation, supernatant and residual reagents were discarded. Six cycles of precipitation and redispersion were repeated. Finally, AgNWs were dispersed in 1-2 mL of ethanol as shown in FIG. 10.

Silver Nanowires Transparent Conductive Ink Formulation:

The ink was formulated by blending purified AgNWs suspended in (MBFLG suspended in ethanol) with PolyBioWire screen printing conductive and transparent water-based ink (www.poly-ink.fr) to obtain formulations comprising 1:1 and 1.5:1 volume ratio, respectively, using a whirl mixer. Inks were blade coated onto glass microscope slides (25×75 mm) with the aid of a polyimide film (Kapton®) at different thickness to control the thickness of the coated electrode. Without any heating treatment or any further processing, the coated electrode films had Rs˜5-8Ω/Square and transparency up to 80-90% at 550 nm as shown in FIG. 11. 4.5 V was the minimum voltage required to generate electrolysis (ROS) based on specific: (1) electrode resistance (Ω/□) value, (2) electrode line width (W) and thickness (T) values, (3) electrodes spacing distance (D) value.

### Example 7—Treatment of Precursor Substrate with Oxygen Plasma or UV Ozone

Oxygen Plasma Treatment:

Oxygen plasma treatments were conducted using an oxygen plasma generation system (such as Mycro, Harrick Plasma) which the working condition can change with power of 10.5-20 W, with 15-40 mL/min oxygen flow, and 1-3 Torr chamber pressure. Different treatment durations can be used, and the treatment time can be varied between 2 min to 30 min. Different ranges of precursor substrates such as plastics, glass, and metal can be used.

UV-Ozone Treatment:

For instance, the precursor substrate sample such as plastic or glass can be put inside the UV/Ozone unit (Jelight Company Inc., USA) which contains a high intensity, low-pressure mercury vapor grid lamp that emits UV light, principally at 184.9 and 253.7 nm wavelengths, which excite molecular oxygen to form atomic oxygen and ozone. In this case, the surface is cleaned, and the surface becomes more hydrophilic when it is oxidised at a distance of 3 cm from the lamp. The exposure time under atmospheric conditions after preheating the UV lamp can be in the range of 20-60 min.

### Example 8—Deposition of a Protective Layer Using Atomic Layer Deposition (ALD)

The surface of metal strips is exposed to oxygen plasma with low pressure of oxygen gas in order to make for an OH group on the surface of metal strips. These —OH group sides can be used as a starting point to form insulating metal oxide such as Al₂O₃ (sequential exposure to dimethylaluminium and water) to form ultra-thin/thin protective layer of Al₂O₃ (in the range 10 nm to 1000 nm). The temperature for the deposition can vary in the range of room temperature to 350 C. However, the temperature can be chosen subject to the type of the substrate and their thermal stability.

This is an example and can be extended in using different coating techniques such as PVD, Inkjet printing, and solution casting, and using different non-conductive stable materials such as metal oxides.

### Example 9—Preparation of a Multi-Layer Structure

To form a multi-layer structure, there are different methods that can be used such as PVD, ALD and inkjet printing.

For instance, a hydrophilic layer can be formed via use of the ink at inkjet printing on the specific substrate such as plastic or glass. The process of coating layers with inkjet printing is explained in Examples 1, 2, 3 and 4. These formed layers can be heated up in order to remove their solvent. The second layer with use of an orthogonal solvent can be used to be deposited by inkjet printing in order to form a multilayer structure.

These multilayer structures can be formed via use of other aforementioned methods such as PVD and ALD which can be used to make multi-layer structures. These can be done via sequential deposition of each metal oxide in these systems.

### Example 10—Deposition of an Antimicrobial and/or Antiviral Film Layer According to the Invention on a Screen

There are different methods of screen printing/coatings. The procedure explained below provides an example in which a PET film was printed using conductive material ink.

For inkjet printing of conductive materials, an A4 polyethylene terephthalate (PET) film, 105 μm thick, and soda-lime glass plate microscope slides 26×76 mm is used. Printing is carried out using a cartridge, in which 8 mL of the conductive ink is preliminarily placed. The print design (which is already stored on the computer drive) is chosen on the desktop and the printer driver settings ticked to produce the maximum printing quality. Printing is performed on the same selected substrate by a single-pass method. After washing, the cartridge is filled with conductive inks without any modification. The design of the printer and the cartridge remains unchanged after the cartridge refill.

### Example 10—Use of Laser Ablation or Cutting System

One method in which conductive layers can be produced on a non-conductive film involves producing the grid like patterns on a PET/non-conductive film which is coated or contains a film of conductive material on it using high precision laser ablation systems. For example, an adhesive aluminium foil can be adhered on top of a non-conductive flexible adhesive film and entered into a roll-to-roll laser ablation system which uses beams of laser with a specific beam length and wavelength to produce the specific pattern on the aluminium foil. The final product contains tracks of conductive materials adhered on the non-conductive substrate.

### Example 11—Use of Photolithography

Photolithography is the process of transferring geometric shapes on a mask to the surface of a silicon wafer. The steps involved in the photolithographic process are wafer cleaning; barrier layer formation; photoresist application; soft baking; mask alignment; exposure and development; and hard-baking.

In the first step, the substrates are chemically cleaned to remove particulate matter on the surface as well as any traces of organic, ionic, and metallic impurities. After cleaning, photoresist is applied to the surface of the substrate. High-speed centrifugal whirling of substrate is the standard method for applying photoresist coatings in micrometre size features on the substrate. This technique, known as “Spin Coating,” produces a thin uniform layer of photoresist on the substrate surface. Positive and Negative Photoresist are two types of photoresist used in photolithography. For positive resists, the resist is exposed with UV light wherever the underlying material is to be removed. In these resists, exposure to the UV light changes the chemical structure of the resist so that it becomes more soluble in the developer. The exposed resist is then washed away by the developer solution, leaving windows of the bare underlying material. In other words, “whatever shows, goes.” The mask, therefore, contains an exact copy of the pattern which is to remain on the substrate. Negative resists behave in just the opposite manner Exposure to the UV light causes the negative resist to become polymerized, and more difficult to dissolve. Therefore, the negative resist remains on the surface wherever it is exposed, and the developer solution removes only the unexposed portions. Masks used for negative photoresists, therefore, contain the inverse (or photographic “negative”) of the pattern to be transferred.

### Example 12—Testing of Antiviral Properties

The antiviral properties of a weak electric voltage were investigated on a murine model of coronavirus which has a very similar structure to Sars-Cov-2 using electric circuits and cell viability assays.

Virus Model:

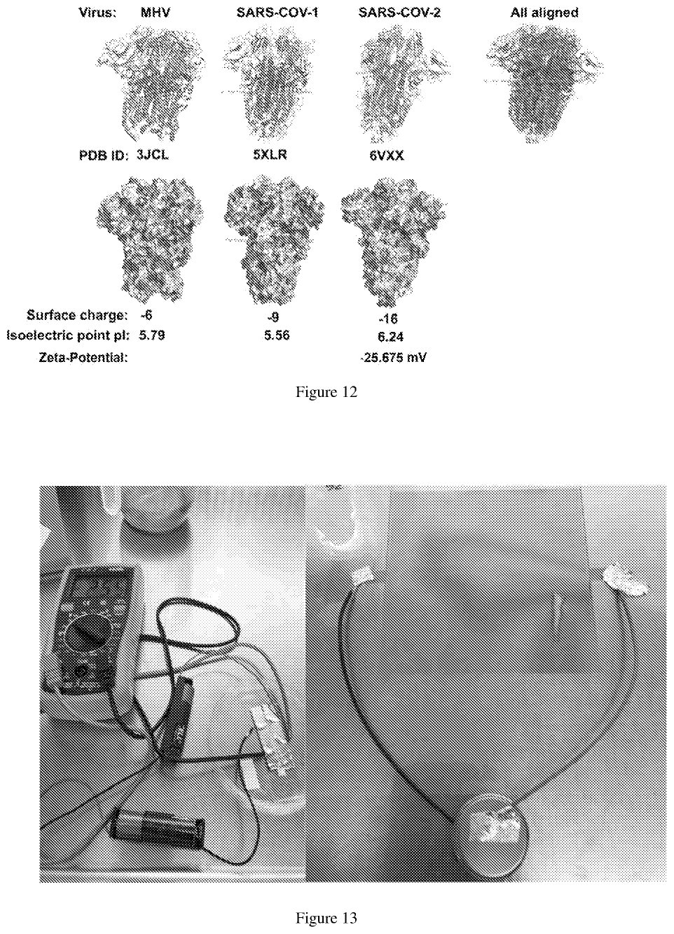

The present experiments use a murine hepatitis virus (MHV, purchased from ATCC® VR764™) which is a group 2 coronavirus (CoV) with a positive-strand RNA virus which has a very similar structure of surface proteins to Sars-CoV-2 which is the virus responsible for Covid-19 pandemic. FIG. 12 provides a comparative analysis of the virus structure, and the surface protein charges between MHV and Sars-CoV-2.

Design Configurations for Testing Antiviral Properties of Weak Electric Voltage:

A capacitor circuit design was created by placing electrodes sidewise and the virus solution was placed between the electrodes for a duration of 60 s or 30 s at 2.55V. For sham control, the virus solution was placed between the electrodes and recovered after the 60 s or 30 s with no electric field applied. FIG. 13 shows the design configurations used for testing MHV.

Cell Viability Assay:

Day 1—Plating Cells:
1. For each experiment the number of required wells of a 96 well plate was calculated depending on the number of samples.
2. L929 cells were collected from T75 flask
   Cell splitting protocol was obtained in suspension
3. Cells were counted, then resuspended at 5×10⁵ cells/ml in complete DMEM (cDMEM)
4. Cells were added in suspension per well (50,000 cells per well) in clear-sided 96-well plate
5. The plate was incubated overnight at 37 C (˜18 h)
6. Should be ˜100,000 cells per well for infection next morning

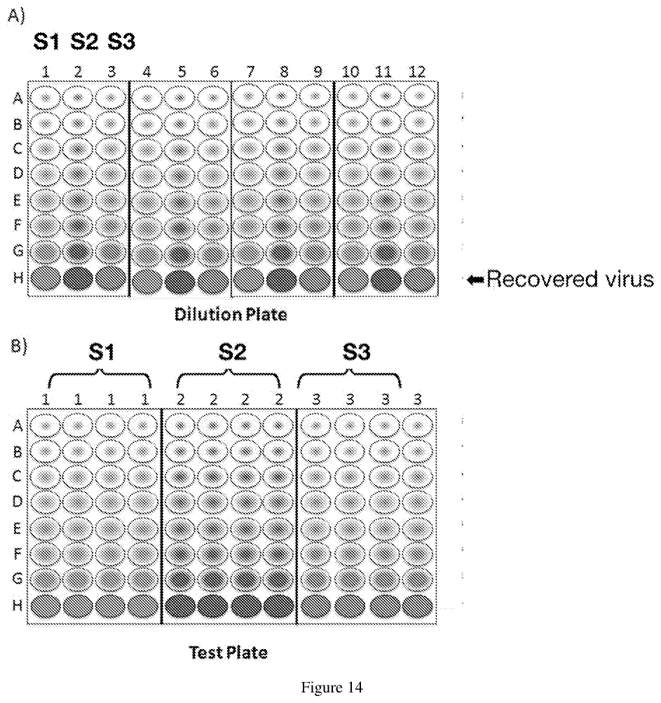

Day 2—Treatment and Infection:
1. cDMEM was added to a test 96 well plate ready for serial dilutions; 20 ul×7 (one column) per sample to be tested
   Described/illustrated in the standard TCID50 protocol and also in FIG. 14.
2. In order to reduce the fizzing effect, the neat virus (MOI of 30 for 10000 cells) solution was diluted with PBS 10 times to produce MOI of 3.
3. The virus was incubated on the material to be tested
   20 ul of virus solution was added to each sample and incubated for the duration of the contact time (60 s or 30 s).
   The treated viruses were then recovered and added to an Eppendorf tube containing 20 ul of the cDMEM.
   4 replicates per treatment (sample) was performed.
4. The treated/untreated viruses were serial diluted by adding 5 ul of treated virus to the 2nd bottom row of the dilutions; mixed well, then 5 ul of the 2nd bottom row were taken and added to the next row. Tips were changed and then mixing and transferring to the next row was repeated for 8 concentrations.
   This gives a 5-fold dilution
   FIG. 14 shows how this was done using the dilution plate
5. The media was removed from cells in the test tube and 20u of fresh media was added to each well.
6. 10 ul of serially diluted MHV or control from the dilution plate was directly added onto cells+media in clear-sided plate (‘test plate’) in quadruplicate (will be 30 ul per well when finished) mixing by pipetting gently.
   Again described/illustrated in the standard TCID50 protocol
7. After one hour, 50 ul of extra fresh media was added to each well.
8. The test plates were then incubated for 48 hours.

Day 4—Reading Results:

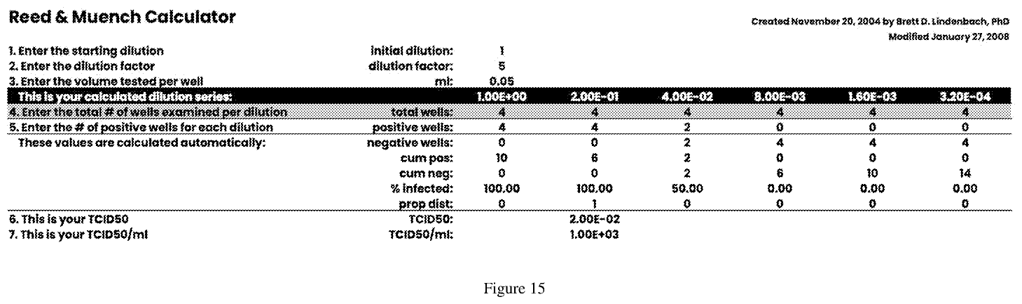

After 48 hours, individual wells in the test plates were observed under the microscope and based on the presence or absence of the cytopathic effect, each well was either recorded as positive (CPE present, infected) or negative (no CPE, healthy). Once the whole plate was observed, the TCID50/ml for each sample was calculated using the Reed-Muench Calculator as shown in FIG. 15. The log reduction value for each sample compared the initial virus TCID50/ml value was then calculated and plotted.

Results:

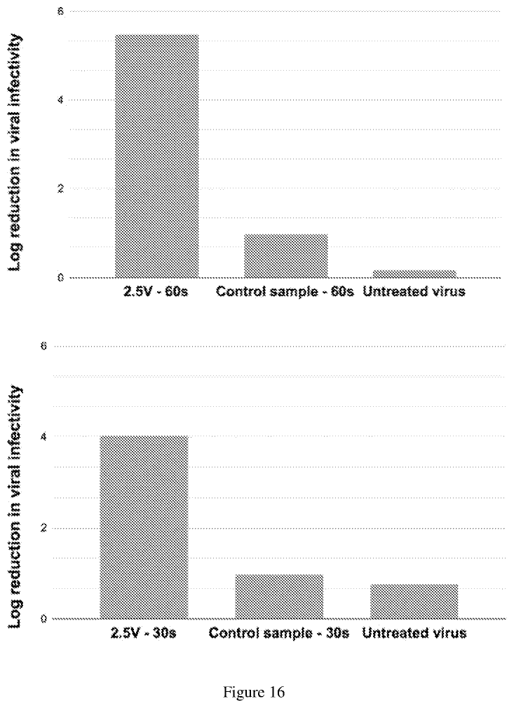

Exposure of MHV to 2.55V electric field in the proposed design resulted in 5.5 and 4 log reduction in CPE after 60 and 30 seconds contact time respectively. The log reduction in untreated viruses and control conditions were less than 1 log in all conditions. These results are demonstrated in FIG. 16.

Discussion:

One interesting observation in these experiments was the fact that the virus solution started to electrolyse when exposed to the electric field present between the electrodes. This effect could be observed by fizzing of the solution which started several seconds after field exposure. This was expected as the virus solution which was dissolved in media or PBS mostly contains water and water electrolysis takes place at a threshold voltage of approximately 1.23V. The electrolysis of water produces a strong antiviral and/or antimicrobial effect.

The inventors of the present invention have surprisingly discovered that the water content of the virus, bacterium, or fungus droplets which land on a surface with a weak electric voltage, itself turns into a disinfecting liquid which inactivates the virus, bacterium, or fungus.

### Example 13—Anti-Viral Effect of the Coating Against Human Coronavirus NL63 at a Contact Time of 10 Sec Relative to a Reference Control

Methods:

For the assay to be valid, the material tested must have no cytotoxic activity on the cells used to quantify the virus, nor interfere with cell sensitivity to infection. The two tests of these criteria are described below.

Cytotoxicity Control: Is the Tested Material Cytotoxic to the Assay's Host Cells?

Assay media was added to treated material and reference control for 5 min, before being collected and added onto monolayers of cells seeded into the wells of a 96-well plate. Cells were then cultured and, after 10 days, a viability assay (crystal violet staining) was used to determine cell viability. The test is carried out in triplicate for both the treated material and nontreated reference control. Media that has been in contact with neither the treated material nor reference control is included as a reference. For the test to be valid, no cytotoxic effect should be observed compared to the media.

Sensitivity Control: Do the Tested Materials Affect the Assay Cells' Sensitivity to the Virus?

Assay media was added to treated material and reference control for 5 min, before being collected in tubes. Next, to test whether exposing the media to the materials affects the cells' sensitivity to infection, 1×10⁵ infectious units of virus were added into each tube. After a 30-min incubation at room temperature, the amount of infectious virus in each sample is quantified (TCID50 assay). The 50% tissue culture infectious dose (TCID50) was the end-point virus dilution where 50% of the infected test cells die. The tests were carried out in triplicate on treated and non-treated material. Media that had not been in contact with either material was also incubated with the virus.

The device of the invention tested here had a substrate of PEN (Polyethylene naphthalene). The conductive material and wire connected to the power supply were both mixtures of conductive inks (60% silver/40% carbon) which were screen printed onto the PEN film. The dimensions of the conductive coating were 8 cm by 8 cm.

Antiviral Test Procedure:

Treated and non-treated materials were placed in individual discs in triplicate. A liquid volume (200 μl) of an appropriate concentration of virus (1×10⁵ PFU/ml stock of human coronavirus NL63) was added onto each surface and covered with an inert film. A lid was placed over each disc, which was then incubated for 10 sec at room temperature in a humidified chamber. At the end of the incubation, the film was lifted, and the sample washed with media to recover the virus. The amount of infectious virus recovered from each sample was then quantified by TCID50. As a further control, virus was added to three pieces of the reference control material and immediately recovered by washing (referred to as the ‘virus recovery control’ or ‘back-titration’). This recovered virus was used to quantify the starting amount of virus.

TCID50 Determination:

A seven-point, ten-fold serial dilution from the virus-containing wash media was tested in quadruplicate for each sample on Rhesus Monkey Kidney Epithelial (LLC-MK2) cells. After 10 days, a viability crystal violet assay was carried out to determine cell viability across the dilution series. The dilution at which 50% of cells are infected/killed (TCID50) was calculated using a regression analysis.

Quantification of Antiviral Activity:

When the test is deemed valid, the antiviral activity (R) is calculated as follows:

R=Ut−At

where
Ut is the average of the common logarithm of the number of infectious units recovered from the untreated test specimens at the end of the incubation time; and
At is the average of the common logarithm of the number of infectious units recovered from the treated test specimens at the end of the incubation time.

An R value of ≥1 indicates antiviral activity.

In this protocol, a pre-determined concentration of virus was dispensed onto test and reference surfaces and incubated at room temperature for 10 sec in a humidified chamber.

Next, the samples were recovered by washing with media (neutraliser), and the amount of infectious virus in each suspension was quantified using a TCID50 assay. For the assay to be valid, the material tested must have no cytotoxic activity on the cells used to quantify the virus, nor interfere with cell sensitivity to infection.

Results:

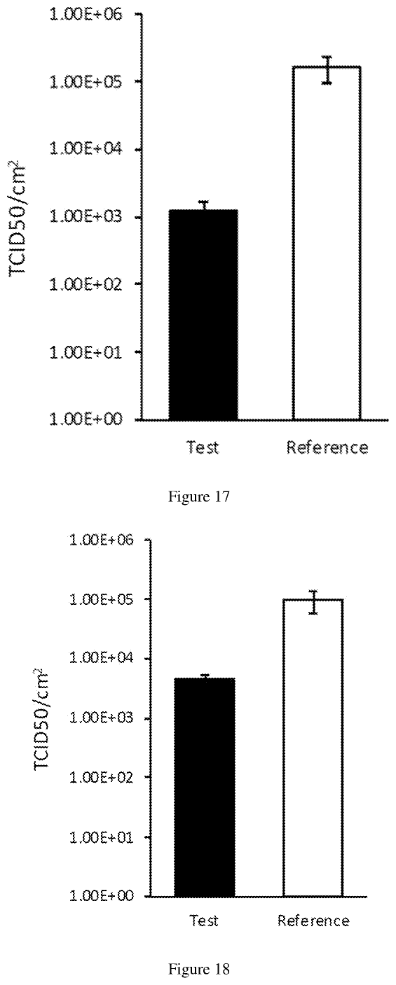

Virucidal activity against human coronavirus NL63 was observed for the treated material relative to the reference control when using a contact time of 10 sec (see FIG. 17). The average recovered titre for the treated material was 1.21E+03 TCID50/cm² compared to the average recovered titre of 1.61E+05 TCID50/cm² for the non-treated reference control. The R value (antiviral activity) was 2.12. Under the conditions tested, the device of the invention displayed virucidal activity against human coronavirus NL63.

TABLE 1
The average infectious units per cm² recovered
from the test and reference control materials
at a contact time of 10 sec with the virus.

| Test | Virus recovery control | Antiviral test |
|---|---|---|
| condition | (TCID50/cm²) | (TCID50/cm²) |
| Test | NA | 1.21E+03 ± 4.57E+02 |
| Reference | 1.29 ± 3.80E+04 | 1.61E+05 ± 6.77E+04 |

TABLE 2
The average infectious units per cm² recovered
from the test and reference control materials
at a contact time of 10 sec with the virus.

| Test condition | TCID50 (log10) | R value | % reduction |
|---|---|---|---|
| Test | 3.08 | 2.12 | 99% |
| Reference | 5.21 | | |

Cytotoxicity:

TABLE 3
Cell viability (%) upon incubation with media recovered from reference
and treated materials, relative to the fresh media control.

| Test condition | Cytotoxicity |
|---|---|
| Test | Not cytotoxic |
| Reference | Not cytotoxic |
| Media | Not cytotoxic |

Sensitivity Control:

Infectious TCID50/cm² recovered after 30 min incubation with 5 ml of media that has been in contact with the treated or untreated material. The difference between the natural logarithm of the infectivity titre of virus recovered from the media only control and each specimen should be less than or equal to 0.5.

TABLE 4

| | | Sensitivity | |
|---|---|---|---|
| Test | Sensitivity control | control | Media - material |
| condition | (TCID50/cm2) | (Log10) | (Log10) |
| Test | 1.33E+05 ± 6.79E+04 | 5.12 | 0.04 |
| Reference | 8.70E+04 ± 3.57E+04 | 4.94 | 0.22 |
| Media | 1.45E+05 ± 6.09E+04 | 5.16 | NA |

Conclusion:

The device tested had no cytotoxic activity towards the cells used to quantify the virus. No cytotoxic activity was detected for the reference control sample. No cytotoxic activity was detected for the media only sample.

### Example 14—Anti-Viral Effect of the Coating Against Human Coronavirus NL63 at a Contact Time of 2 Sec Relative to a Reference Control

This study tested the antiviral activity of the same device as that used in Example 15 against human coronavirus NL63 at a contact time of 2 sec relative to a non-treated reference control.

Experimental protocol was as for Example 15 in that a pre-determined concentration of virus was dispensed onto test and reference surfaces and incubated at room temperature for 2 sec in a humidified chamber.

Next, the samples were recovered by washing with media (neutraliser), and the amount of infectious virus in each suspension was quantified using a TCID50 assay. For the assay to be valid, the material tested must have no cytotoxic activity on the cells used to quantify the virus, nor interfere with cell sensitivity to infection.

Results:

Virucidal activity against human coronavirus NL63 was observed for the treated material relative to the reference control (see FIG. 18). The device displayed antiviral activity against human coronavirus NL63. The average recovered titre for the treated material was 4.50E+03 TCID50/cm² compared to the average recovered titre of 9.67E+04 TCID50/cm2 for the non-treated reference control. The R value (antiviral activity) was 1.33. Under the conditions tested, the device displayed virucidal activity against human coronavirus NL63.

TABLE 5
The average infectious units per cm² recovered
from the test and reference control materials
at a contact time of 2 sec with the virus.

| Test | Virus recovery control | Antiviral test |
|---|---|---|
| condition | (TCID50/cm²) | (TCID50/cm²) |
| Test | NA | 4.50E+03 ± 7.70E+02 |
| Reference | 1.29E+05 ± 3.80E+04 | 9.67E+04 ± 3.96E+04 |

TABLE 6
The average infectious units per cm² recovered
from the test and reference control materials
at a contact time of 2 sec with the virus.

| Test condition | TCID50 (log10) | R value | % reduction |
|---|---|---|---|
| Test | 3.65 | 1.33 | 90% |
| Reference | 4.99 | | |

Cytotoxicity:

TABLE 7
Cell viability (%) upon incubation with media recovered from reference
and treated materials, relative to the fresh media control.

| Test condition | Cytotoxicity |
|---|---|
| Test | Not cytotoxic |
| Reference | Not cytotoxic |
| Media | Not cytotoxic |

Sensitivity Control:

Infectious TCID50/cm² recovered after 30 min incubation with 5 ml of media that has been in contact with the treated or untreated material. The difference between the natural logarithm of the infectivity titre of virus recovered from the media only control and each specimen should be less than or equal to 0.5.

TABLE 8

| | | Sensitivity | |
|---|---|---|---|
| Test | Sensitivity control | control | Media - material |
| condition | (TCID50/cm2) | (Log10) | (Log10) |
| Test | 1.33E+05 ± 6.79E+04 | 5.12 | 0.04 |
| Reference | 8.70E+04 ± 3.57E+04 | 4.94 | 0.22 |
| Media | 1.45E+05 ± 6.09E+04 | 5.16 | NA |

Conclusion:

Based on the findings reported here and following ISO 21702, the treated material displays virucidal activity against human coronavirus NL63 after a contact time of 2 sec. The test material had no cytotoxic activity towards the cells used to quantify the virus. No cytotoxic activity was detected for the reference control sample. No cytotoxic activity was detected for the media only sample.

### Example 15—Antiviral and Antibacterial Coatings for Touch Screens

Antiviral and antibacterial coatings on transparent glass substrates suitable for use as touch screens have been fabricated and tested. Transparent coatings were created using photolithography followed by wet etching, as well as lift-off processes and micro imprinting methods.

Photolithography

Photolithography was performed on a commercially available glass substrate (Borofloat® 33) and on tempered glass screen protectors. Before performing lithography, the glass substrates were thoroughly cleaned using Decon® 90 and then, to remove any organic residue, wafers were dry cleaned in oxygen plasma using 300 W power and a gas flow rate of 10 sccm for 15 minutes. The photosensitive resist AZ-5214E was then spun-coated by spinning at 4000 rpm for 60 seconds. To remove the solvent, the wafer and the resist were baked at 110° C. for two minutes.

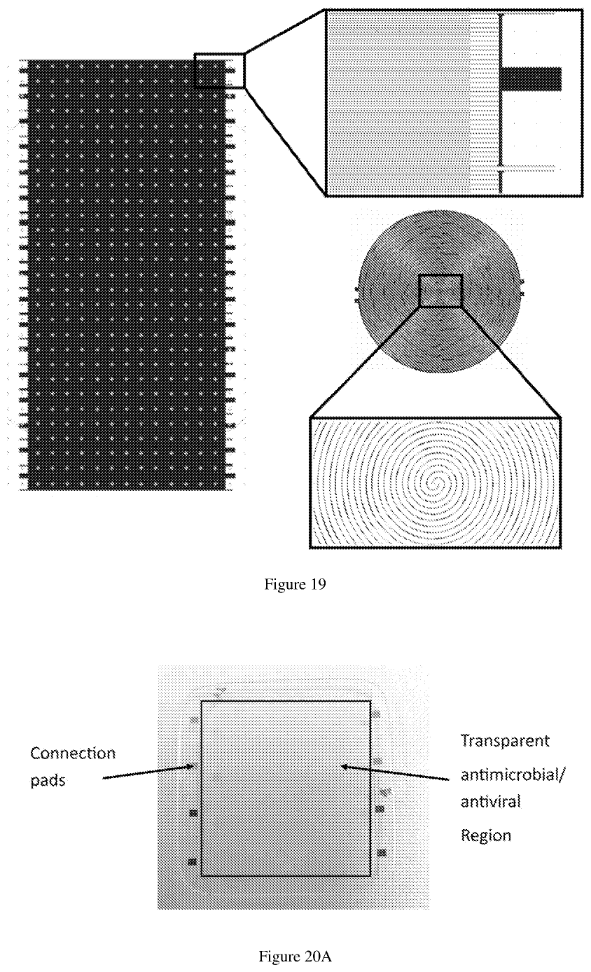

The photoresist on the wafer was then exposed using a direct-write contactless optical lithography laser, (Microtech LW-405B+). Different electrode designs were tested. FIG. 19 illustrates the parallel, as well as circular, interdigitated electrodes used in this experiment.

To perform laser-based lithography, a laser with wavelengths of 375 and 405 nm was used, and the resist was exposed using optical resolution by replacing the final focusing lens 0.8 and 8 μm which exposed the resist to 261 mJ/cm². The laser wavelength was 405 nm using a 30% filter. The substrate was centred and three points in opposite corners were set to the right focus and set up a plane to focus throughout the sample to write with the right height (z). The exposure time for a watch sized screen was 45 minutes, whereas exposure time for a mobile phone sized screen was 240 minutes.

It is of note that laser lithography is a serial exposure technique and so is slow. However, it provides flexibility in changing and optimising the design. It will be appreciated that the fabrication process may alternatively use other techniques such as projection photolithography which is a parallel and faster technique.

Having exposed the pattern on the resist layer using laser lithography, the exposed regions of the resist were developed using photoresist developer solution, 1:4 AZ-351B/DI water, resulting in a surface having exposed regions with a width of 5 μm and a gap of 95 μm between each exposed region.

Electron-Beam Metal Evaporation:

A 20 nm of Titanium (Ti) layer followed by 130 nm of gold (Au) layer was deposited using an electron-beam (e-beam) evaporator. Having loaded the sample into the vacuum chamber, the chamber was pumped below 10⁻⁶ Torr and the cryopump temperature was below 20K. Having reached the base pressure, the Ti layer was deposited at a rate of 1 Å/s, followed by the gold layer that was evaporated at a rate of 2 Å/s. The distance between the metal source and the substrate was 49 cm. The sample was then taken out of the vacuum chamber for the lift-off process.

Lift-Off Process:

The lift-off process was performed to remove the Ti/Au layer from unwanted regions. i.e. the areas where Ti/Au layer is deposited on the photoresist. The wafer was soaked in acetone solution for about one hour so that photoresist on the glass substrate was completely dissolved. The Ti/Au layer evaporated on the resist floated into the acetone solution, whereas the regions that were exposed using laser lithography remained attached to the glass substrate, resulting in a desired Ti/Au interdigitated electrode structure on a glass substrate.

FIG. 20 shows an optical image of the array. The black square indicates the antiviral/antibacterial region where interdigitated electrodes are present. The spacing between the electrodes was more than 20 times the width of the Ti/Au electrodes, leading to more than 95% transparency. The connection pads were for testing the electrodes and providing power to the device.

Etching Process:

Arrays have also been fabricated using an etching process. In this process, Ti/Au layers were sputter deposited on a plasma cleaned glass substrate. Photolithography was then performed to create an interdigitated pattern on a gold surface. During this exposure, gold regions that were not desirable were exposed. The substrate was then dipped into a gold etching solution which consisted of potassium iodide. Gold regions that were protected by the resist were not etched and the exposed regions were etched. The titanium layer was not etched by the gold layer. The titanium layer was then etched using a reactive ion etch process, using a 1:1 combination of SF6 and Argon gases.

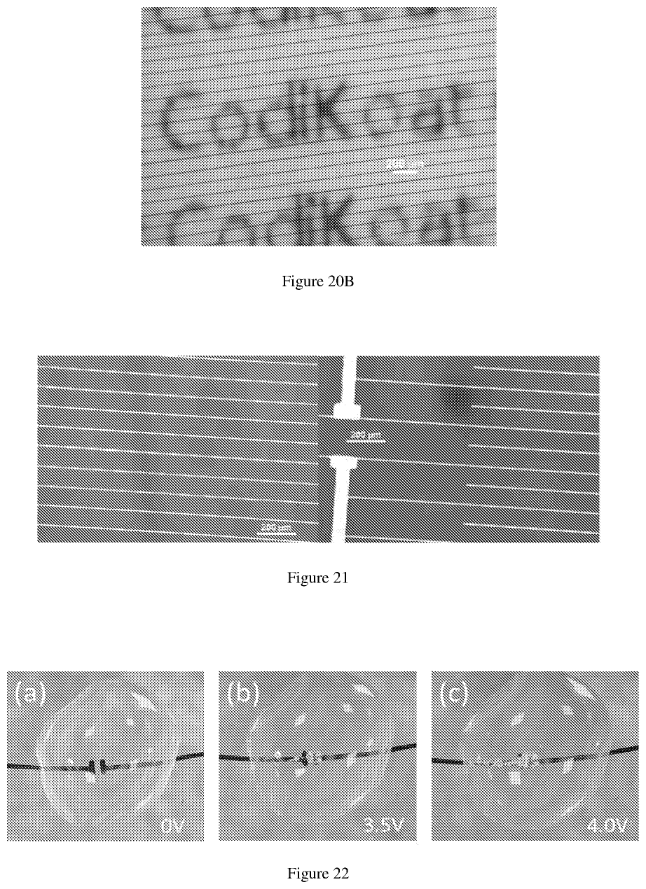

After etching the Ti layer, the resist was removed using a resist remover solution. Micrographs of the electrodes are shown in FIG. 21.

Measurements:

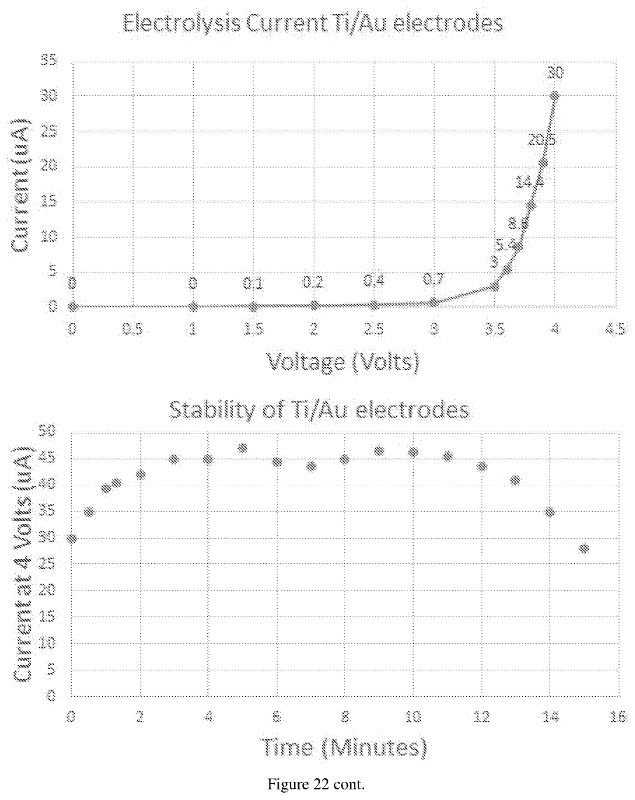

To test the stability of the Ti/Au electrodes, a water droplet of volume 10 μl was placed between the electrodes, as shown in FIG. 22. The voltage was slowly ramped up and the current flow between the electrodes was measured. A rise in current started at 3.5 volts, which is the minimum voltage required to start the electrolysis process. To test the stability, the voltage was increased to 4 volts and then the change in current was observed over time. Within the first few minutes, the current increased from 30 μA to 40 μA and remained stable for 15 minutes. During this time, the droplet started to dry, resulting in a decrease in the current, as can be seen in FIG. 22E.

Visual inspection of the electrodes showed that Ti/Au electrodes are intact. Measurements were replicated at the same location, demonstrating that these electrodes can be reused. In comparison, when Cr/Au electrodes were tested, Chromium (Cr) was etched, and the electrodes were found to peeled off from the glass surface within a few seconds. These measurements demonstrate that Ti is a more reliable material for attaching gold to a glass surface.

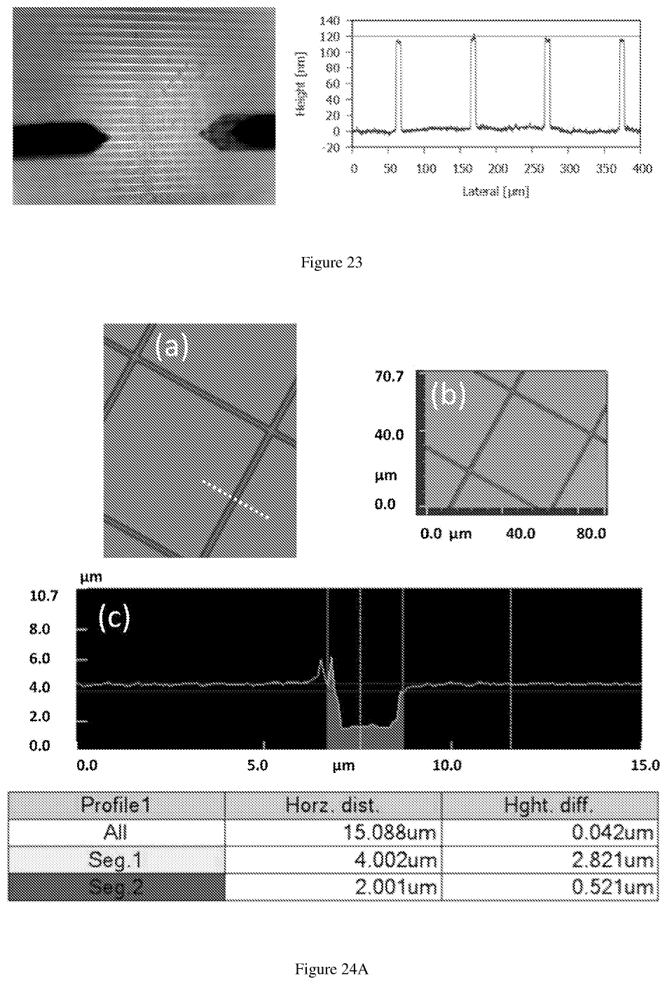

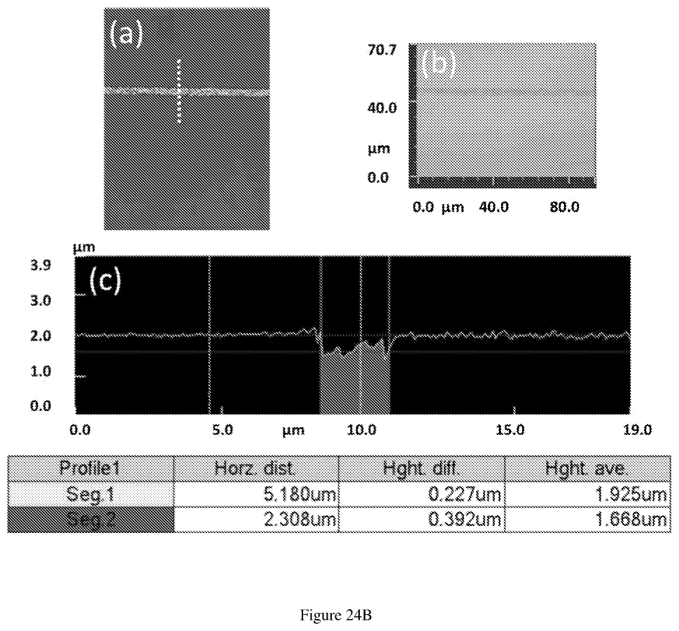

To measure the thickness of the metal electrodes, a sample was measured using a profilometer (Bruker Dektak XT Stylus). The scans were performed using a vertical resolution of 65.5 μm and scan lengths of 400 and 1400 nm. The stylus force was 3 mg. The measurements shown in FIG. 23 confirm that the total height of Ti/Au electrodes was 120 nm and the pitch was 100 um.

During the effervescence or bubbling process, as can be seen in FIG. 22c, water is split into H⁺ and OH⁻. The hydrogen ions (H⁺) gain electrons from the cathode and form hydrogen gas (H₂), which is released as bubbles. Presence of hydroxide ions (OH) makes the water around cathode more alkaline. However, at the anode, hydroxide ions (OH) give electrons to the anode and form oxygen gas (O₂) and hydrogen ions that remain in the solution make it more acidic around the anode. These hydrogen and hydroxide ions present in the solution are lethal to pathogenic viruses and bacteria because the ions react with the lipid envelope and proteins around the pathogens causing the outer membrane to rupture resulting in inactivation of the virus/bacteria.

Imprinting Method:

Transparent conducting films have also been created with an array of silver microwires on a PET flexible substrate, using a high-speed imprinting and ink-filling method. To fabricate these arrays, trenches having a depth of about 2.5 μm were fabricated using micro imprinting and then these trenches were filled with silver ink formulated as described in Example 6. FIG. 24 shows images of the transparent array, the depth of the trenches before (FIG. 24A) and after (FIG. 24B) filling with silver, and analysis of the silver wires. The top two images show that the width of the trenches was about 2 μm. The height profile graph in the middle shows that the depth of the trench was 2.8 μm. The table below summarises the height profile measurements.

1.-79. (canceled)

80. An insulator substrate having an antimicrobial and/or antiviral layer thereon, said layer comprising a conductive material;
wherein the conductive material is connected to a power supply; and
wherein the power supply and the conductive material are arranged such that the power supply is capable of inducing a voltage across the layer, such that a capacitor structure is formed on a surface of the substrate.

81. The insulator substrate according to claim 80, wherein the conductive material is arranged into at least two, or a plurality of, strips on a surface of the substrate;
wherein the strips are spaced apart from each other; and
wherein the strips are alternately connected to positive and negative connections of the power supply, or a photocathode and a photoanode, so as to induce a voltage across the film layer between adjacent strips.

82. The insulator substrate according to claim 80, wherein the voltage is in the range of 1V to 10V, preferably between 1V and 5V.

83. The insulator substrate according to claim 80, wherein the conductive material comprises:
i) at least one metal, optionally selected from silver, aluminium, zinc, copper, titanium, chromium, iron, platinum, palladium, tungsten, and gold, as well as multilayer combinations and alloys thereof;
ii) a metal oxide, optionally selected from nickel-, cobalt- and iron-based oxides, indium tin oxide and fluorinated tin oxide, and mixtures thereof;
iii) photoactive metal nanoparticles, metal oxide nanoparticles, metal sensitised with photoactive dyes, and/or metal oxides sensitised with photoactive dyes; and/or
iv) a doped metal oxide, optionally wherein the doped metal oxide is aluminium doped zinc oxide.

84. The insulator substrate according to claim 80 wherein the conductive material further comprises at least one non-metal, graphite, graphene or carbon, for example carbon nanostructures such as carbon nanotubes, and/or nanoparticles.

85. The insulator substrate according to claim 80, wherein the conductive material comprises a single layer or multilayers of conductive material.

86. The insulator substrate according to claim 80, wherein the conductive material is formulated as an ink.

87. The insulator substrate according to claim 80, wherein the antimicrobial and/or antiviral layer is substantially transparent, semi-transparent, or has an overall transparency of between about 50% and 99% in the visible spectrum, optionally wherein the antimicrobial and/or antiviral layer is made of nickel-, cobalt- and iron-based oxides, indium tin oxide, fluorinated tin oxide, aluminium doped zinc oxide.

88. The insulator substrate according to claim 80, wherein the antimicrobial and/or antiviral layer is substantially opaque, optionally wherein the antimicrobial and/or antiviral layer is made of silver, aluminium, zinc, copper, titanium, iron, or gold.

89. The insulator substrate according to claim 80, wherein the conductive material is arranged in strips having a width that is between about 2 to 100 times smaller than the distance between the conductive material strips.

90. The insulator substrate according to claim 80, wherein the conductive material is arranged in strips and the distance between each adjacent strips on the surface of the substrate independently is in the range of 0.5 μm to 1 cm, preferably in the range of 2 μm to 20 μm, more preferably in the range of 2 μm to 300 μm.

91. The insulator substrate according to claim 80 wherein the conductive material is arranged in strips and the width of each strip independently is in the range of 0.5 μm to 1 cm, preferably in the range of 2 μm to 500 μm, more preferably in the range of 2 μm to 300 μm, or wherein the strips have a width of between about 0.01 mm and 10 nm, or in a range of between about 1 μm and about 5 μm, or in a range of between about 10 μm and about 200 μm, optionally having a width of less than about 5 μm.

92. The insulator substrate according to claim 80, wherein the conductive material is arranged in strips each having a thickness in the range of 5 nm to 1000 μm, preferably in the range of 500 nm to 500 μm, more preferably in the range of 1 μm to 100 μm, or in a range of between about 20 nm and about 500 nm, or in a range of between about 1 μm and about 10 μm.

93. The insulator substrate according to claim 80, wherein the conductive material is arranged in strips which are interdigitated.

94. The insulator substrate according to claim 80, wherein the antimicrobial and/or antiviral layer further comprises a hygroscopic component, optionally selected from rock salt, silica sand, silica gel, CaCl₂, a hygroscopic polymer, or a glycosaminoglycan.

95. The insulator substrate according to claim 94, wherein the hygroscopic component comprises an aqueous component, preferably water.

96. The insulator substrate according to claim 80, wherein the antimicrobial and/or antiviral layer is partially or fully covered by a protective layer, optionally selected from epoxy or any non-conductive metal oxide such as aluminium oxide or silicon dioxide.

97. The insulator substrate according to claim 80, wherein the insulator substrate has a surface beneath the antimicrobial and/or antiviral layer that has been pre-treated with oxygen plasma or UV ozone, such that the surface tension of the surface of the insulator substrate is reduced.

98. The insulator substrate according to claim 80, wherein the power supply is a solar cell, optionally wherein the solar cell is an organic or amorphous silicon solar cell, preferably wherein the solar cell is an OPV (organic photovoltaic), a DSSC (Dye-sensitised solar cells), a copper indium gallium selenide (CIGS) solar cell, or a flexible silicon solar cell.

99. The insulator substrate according to claim 80, wherein the power supply is a battery, optionally wherein the battery has a thickness in the range of 1 mm to 10 mm, or in the range of 10 μm to 100 μm.

100. The insulator substrate according to claim 80, wherein the conductive material is connected to the power supply by a wire, the wire comprising one or more of:
i) a metal;
ii) a conductive metal oxide;
iii) a doped metal oxide; and/or
iv) graphene or carbon, for example carbon nanostructures such as carbon nanotubes.

101. The insulator substrate according to claim 80, wherein the insulator substrate is flexible, optionally wherein the insulator substrate is a polymeric adhesive film, fabric, plastic, paper, fibres, or amorphous silicon.

102. The insulator substrate according to claim 80, wherein the insulator substrate is non-flexible, optionally wherein the insulator substrate is glass, plastic, or ceramic.

103. An article of manufacture comprising the insulator substrate according to claim 80.

104. The article of manufacture according to claim 103, wherein the article of manufacture is selected from an electronic device such as a tablet or mobile phone, a touchscreen, a keypad, a keyboard, a button, a handle, a screen protector, a piece of furniture such as a seat, a table, an arm rest, a toilet seat, a cup, a worktop, and an appliance, such as a kitchen appliance, a door plate, a water tap, a car steering wheel, a car key, food packaging, stationary, and a bank note.
